(19) United States
(12) Patent Application Publication
(10) Pub. No.: US 2021/0023181 A1
(43) Pub. Date: Jan. 28, 2021

(54) USE OF AXL, CCL19 AND/OR BMP-6 FOR PROMOTING WOUND HEALING

(71) Applicant: IMPERIAL COLLEGE OF SCIENCE, TECHNOLOGY AND MEDICINE, London (GB)

(72) Inventors: Helena Topouzi, London (GB); Claire Higgins, London (GB)

(73) Assignee: IMPERIAL COLLEGE OF SCIENCE, TECHNOLOGY AND MEDICINE, London (GB)

(21) Appl. No.: 16/982,222

(22) PCT Filed: Mar. 19, 2019
(86) PCT No.: PCT/GB2019/050763

§ 371 Date: Sep. 18, 2020

(30) Foreign Application Priority Data
Mar. 19, 2018 (GB) 1804355.4

(51) Int. Cl. A61K 38/45; A61K 38/19; A61P 17/02

(52) U.S. Cl. CPC A61K 38/45; A61P 17/02; A61K 38/19; A61K 38/195

(57) ABSTRACT

The invention relates to the treatment of a wound, and in particular to uses of polypeptides (or genetic constructs or vectors encoding such peptides) to promote wound healing and/or reduce, prevent or inhibit scarring. The invention extends to pharmaceutical compositions comprising such polypeptides or constructs, for treating wounds, and for reducing scarring, and cosmetic formulations for improving the appearance of skin. The invention also extends to wound dressings, formulations and bandages comprising such polypeptides.

The present invention relates to the treatment of a wound, and in particular to uses of polypeptides (or genetic constructs or vectors encoding such peptides) to promote wound healing and/or reduce, prevent or inhibit scarring. The invention extends to pharmaceutical compositions comprising such polypeptides or constructs, for treating wounds, and for reducing scarring, and cosmetic formulations for improving the appearance of skin. The invention also extends to wound dressings, formulations and bandages comprising such polypeptides.

Skin is a segmented structure composed of epidermal, dermal and hypodermal layers. However, this is an oversimplified way of describing the integument, as each segment has its own complexity that contributes to the maintenance of organ homeostasis. Multi-layered, stratified keratinocytes comprise the majority of cells within the epidermis; the interface of the body with the external environment. The underlying dermis is predominantly composed of connective tissue containing fibroblasts, blood vessels, nerves and immune cells (1, 2), while deeper still is the hypodermis, a layer of adipocytes with a role in metabolic homeostasis (3, 4).

As the largest organ in the body, our skin provides us with external protection, and internal homeostasis. Non-healing skin wounds account for 2-4% of the health care budget in industrialised counties, with 1-2% of the population affected by such a wound at any time (5). Specifically in the UK, the burden of treating chronic wounds and associated co-morbidities costs the NHS in excess of 5 billion pounds per year (6), which is higher than the costs of treating obesity. In addition, harbouring a chronic wound has both a psychological and physical impact, negatively affecting quality of life of patients (7, 8). While some products such as Regranex (PDGF-BB) were marketed to promote healing of full-thickness wounds, the EMA withdrew their marketing authorisation of Regranex in 2012 after secondary malignancies became more widespread in patients. Thus, chronic wounds remain very much a major challenge in modern medicine.

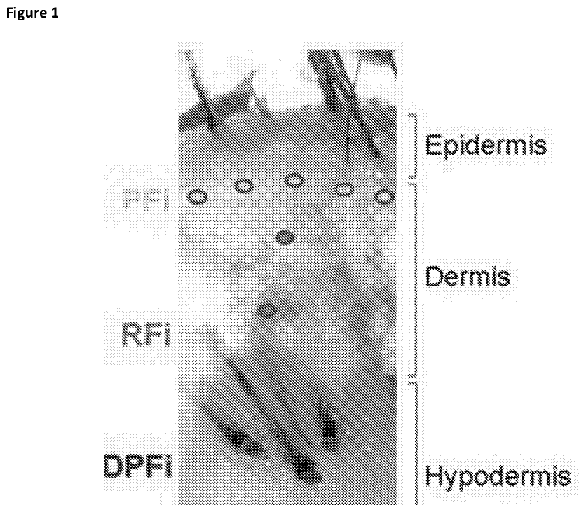

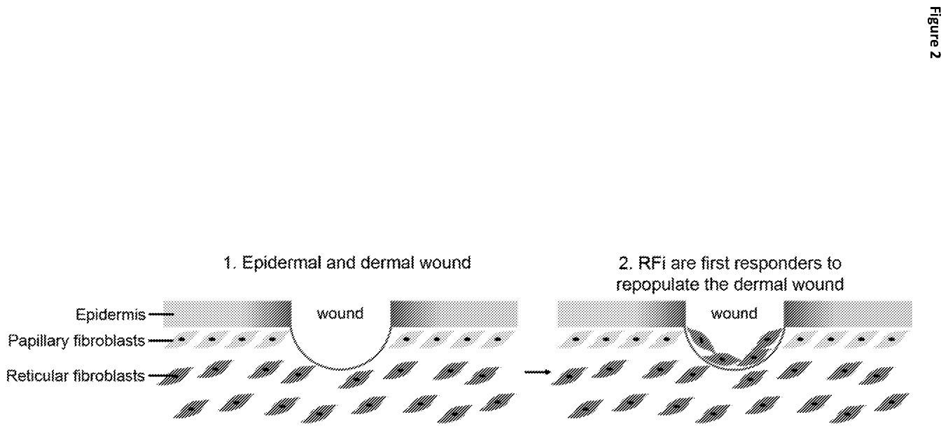

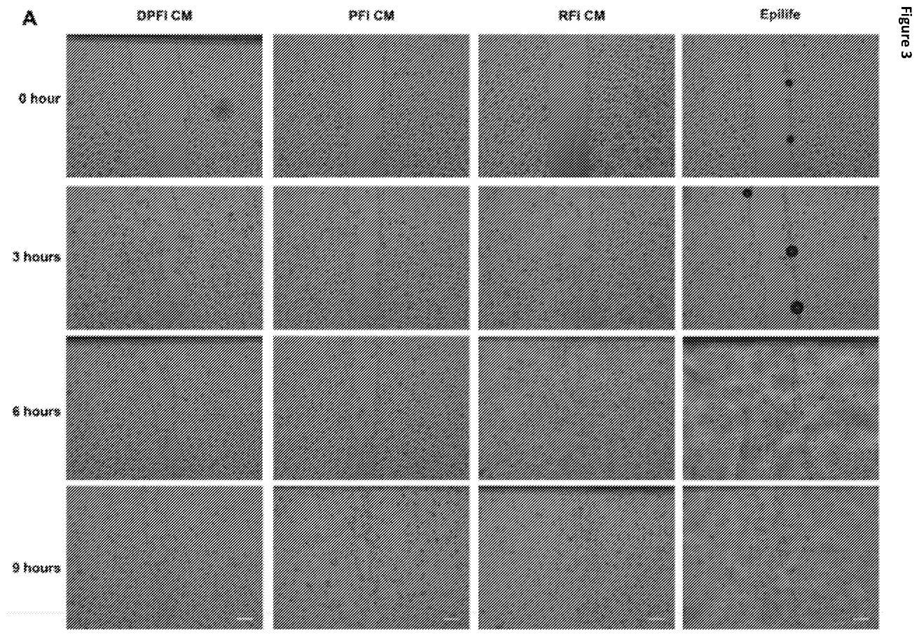

Wound closure is characterised by three phases; 1) re-epithelisation of the wound, 2) dermal matrix deposition, and 3) dermal re-modelling. Migration of epithelial keratinocytes across a wound bed is a key step in wound repair, and as delayed re-epithelisation is one of the main factors which leads to development of a chronic wound many therapies seek to direct migration of keratinocytes for wound closure (9). In species which heal quickly without scarring, such as axolotl, re-epithelialisation of the wound is extremely quick, followed by an extended re-modelling phase. In contrast, in chronic wounds, re-epithelisation does not occur, and subsequent re-modelling is also perturbed. In skin homeostasis, dermal fibroblasts are the conductors that orchestrate the migration and differentiation of keratinocytes in the overlying epithelium (10). The term fibroblast is usually used to refer en masse to all cells regardless of their sub-anatomical location with the skin. However, fibroblasts from different depths in the dermis, and both inside and outside the hair follicle have different lineages and behaviours (1, 11). Fibroblasts in the dermis closest to the epithelium are termed papillary fibroblasts (PFi), while those in the lower interfollicular dermis are referred to as reticular fibroblasts (RFi) (see FIG. 1). At the base of the hair follicle, there are dermal papilla fibroblasts (DPFi) while dermal sheath fibroblasts (DSFi) wrap around the follicle exterior. Lineage tracing studies of these fibroblast sub-types after murine skin injury revealed that RFi are the first fibroblasts to migrate into the wound after injury (12) (see FIG. 2).

Scarring is an inherent human property, which occurs due to impaired dermal re-modelling in the third phase of wound closure. However, chronic wounds with delayed re-epithelisation are characterised by extensive scarring, and there are clear links between scar formation and the time it takes for the wound to initially close. For example, re-epithelisation also occurs faster in oral wounds compared to skin wounds, and oral scars are few and far between. Thus, it is well accepted that wounds which close with faster re-epithelisation will have smaller scars.

There is therefore a need to provide improved wound treatment compositions, in particular to provide an increase in the rate of wound healing and/or to prevent, reduce or inhibit scar formation.

The inventors have characterised specific sub-populations of fibroblasts that are believe to be key players in wound healing, which release factors which have a paracrine effect on keratinocytes during wound closure. Specifically, they have shown that dermal papilla (DPFi) would promote faster closure of scratch wounds than papillary fibroblasts (PFi), reticular fibroblasts (RFi) and controls. Accordingly, the inventors have identified polypeptides uniquely produced by DPFi that are surprisingly effective by themselves and in combination with one another in promoting faster wound healing by promoting re-epithelisation. The inventors evaluated re-epithelisation of wounds in scratch assays and in ex vivo human skin biopsies. Furthermore, as chronic wounds have enhanced risk of wound site infection as the barrier function of the skin is no more, the polypeptides of the present invention also help to reduce infection by acceleration re-establishment of the skin barrier. Additionally, the polypeptides described herein promote reduced scar formation in both normal wound closure and chronic wounds.

Hence, according to a first aspect of the invention, there is provided a polypeptide selected from the group consisting of AXL, CCL19 and BMP-6, or a biologically active variant or fragment thereof, for use in treating a wound.

In one embodiment, the polypeptide for use in treating a wound is AXL or a biologically active variant or fragment thereof comprising the active domain of AXL, and preferably the polypeptide is soluble AXL. The skilled person will understand that AXL is also known as UFO, JTK11 or Tyro7.

In another embodiment, the polypeptide for use in treating a wound is BMP-6, or a biologically active variant or fragment thereof.

In yet another embodiment, the polypeptide for use in treating a wound is CCL19, or a biologically active variant or fragment thereof.

In a preferred embodiment, AXL or a biologically active variant or fragment thereof comprising the active domain of AXL is used in combination with CCL19 and/or BMP-6 and/or a biologically active variant or fragment thereof. Most preferably, AXL or a biologically active variant or fragment thereof, is used in combination with CCL19.

One embodiment of the 894 amino acid human polypeptide AXL (NP_068713) is provided herein as SEQ ID NO: 1, as follows:

[SEQ ID NO: 1]
mawrcprmgr vplawclalc gwacmaprgt qaeespfvgn
pgnitgargl tgtlrcqlqv ggeppevhwl rdgqilelad
stqtqvplge deqddwivvs qlritslqls dtgqyqclvf
lghqtfvsqp gyvgleglpy fleepedrtv aantpfnlsc
gaggppepvd llwlgdavpl atapghgpqr slhvpglnkt
ssfsceahna kgvttsrtat itvlpqqprn lhlvsrqpte
levawtpgls giyplthctl qavlsddgmg igagepdppe
epltsgasvp phqlrlgslh phtpyhirva ctssqgpssw
thwlpvetpe gvplgppeni satrngsqaf vhwqeprapl
qgtllgyrla yqgqdtpevl mdiglrqevt lelqgdgsys
nitvcvaayt aagdgpwslp vpleawrpgq aqpvhqlvke
pstpafswpw wyvllgavva aacvlilalf lvhrrkketr
ygevfeptve rgelvvryry rksysrrtte atlnslgise
elkeklrdvm vdrhkvalgk tlgegefgav megglnqdds
ilkvavktmk iaictrsele dflseavcmk efdhpnvmrl
igvcfqgser esfpapvvil pfmkhgdlhs fllysrlgdq
pvylptqmlv kfmadiasgm eylstkrfih rdlaarncml
nenmsvcvad fglskkiyng dyyrqgriak mpvkwiaies
ladrvytsks dvwsfgvtmw eiatrgqtpy pgvenseiyd
ylrqgnrlkg padcldglya lmsrcwelnp qdrpsftelr
edlentlkal ppaqepdeil yvnmdegggy peppgaagga
dpptqpdpkd scscltaaev hpagryvlcp sttpspagpa
drgspaapgq edga

Accordingly, preferably the AXL polypeptide comprises an amino acid sequence substantially as set out in SEQ ID NO:1, or a biologically active variant or fragment thereof.

In one embodiment, AXL may be encoded by a nucleotide sequence (NM_021913) which is provided herein as SEQ ID NO: 2, as follows:

[SEQ ID NO: 2]
ATGGCGTGGCGGTGCCCCAGGATGGGCAGGGTCCCGCTGGCCTGGTGCTT
GGCGCTGTGCGGCTGGGCGTGCATGGCCCCCAGGGGCACGCAGGCTGAAG
AAAGTCCCTTCGTGGGCAACCCAGGGAATATCACAGGTGCCCGGGGACTC
ACGGGCACCCTTCGGTGTCAGCTCCAGGTTCAGGGAGAGCCCCCCGAGGT
ACATTGGCTTCGGGATGGACAGATCCTGGAGCTCGCGGACAGCACCCAGA
CCCAGGTGCCCCTGGGTGAGGATGAACAGGATGACTGGATAGTGGTCAGC
CAGCTCAGAATCACCTCCCTGCAGCTTTCCGACACGGGACAGTACCAGTG
TTTGGTGTTTCTGGGACATCAGACCTTCGTGTCCCAGCCTGGCTATGTTG
GGCTGGAGGGCTTGCCTTACTTCCTGGAGGAGCCCGAAGACAGGACTGTG
GCCGCCAACACCCCCTTCAACCTGAGCTGCCAAGCTCAGGGACCCCCAGA
GCCCGTGGACCTACTCTGGCTCCAGGATGCTGTCCCCCTGGCCACGGCTC
CAGGTCACGGCCCCCAGCGCAGCCTGCATGTTCCAGGGCTGAACAAGACA
TCCTCTTTCTCCTGCGAAGCCCATAACGCCAAGGGGGTCACCACATCCCG
CACAGCCACCATCACAGTGCTCCCCCAGCAGCCCCGTAACCTCCACCTGG
TCTCCCGCCAACCCACGGAGCTGGAGGTGGCTTGGACTCCAGGCCTGAGC
GGCATCTACCCCCTGACCCACTGCACCCTGCAGGCTGTGCTGTCAGACGA
TGGGATGGGCATCCAGGCGGGAGAACCAGACCCCCCAGAGGAGCCCCTCA
CCTCGCAAGCATCCGTGCCCCCCCATCAGCTTCGGCTAGGCAGCCTCCAT
CCTCACACCCCTTATCACATCCGCGTGGCATGCACCAGCAGCCAGGGCCC
CTCATCCTGGACCCACTGGCTTCCTGTGGAGACGCCGGAGGGAGTGCCCC
TGGGCCCCCCTGAGAACATTAGTGCTACGCGGAATGGGAGCCAGGCCTTC
GTGCATTGGCAAGAGCCCCGGGCGCCCCTGCAGGGTACCCTGTTAGGGTA
CCGGCTGGCGTATCAAGGCCAGGACACCCCAGAGGTGCTAATGGACATAG
GGCTAAGGCAAGAGGTGACCCTGGAGCTGCAGGGGGACGGGTCTGTGTCC
AATCTGACAGTGTGTGTGGCAGCCTACACTGCTGCTGGGGATGGACCCTG
GAGCCTCCCAGTACCCCTGGAGGCCTGGCGCCCAGGGCAAGCACAGCCAG
TCCACCAGCTGGTGAAGGAACCTTCAACTCCTGCCTTCTCGTGGCCCTGG
TGGTATGTACTGCTAGGAGCAGTCGTGGCCGCTGCCTGTGTCCTCATCTT
GGCTCTCTTCCTTGTCCACCGGCGAAAGAAGGAGACCCGTTATGGAGAAG
TGTTTGAACCAACAGTGGAAAGAGGTGAACTGGTAGTCAGGTACCGCGTG
CGCAAGTCCTACAGTCGTCGGACCACTGAAGCTACCTTGAACAGCCTGGG
CATCAGTGAAGAGCTGAAGGAGAAGCTGCGGGATGTGATGGTGGACCGGC
ACAAGGTGGCCCTGGGGAAGACTCTGGGAGAGGGAGAGTTTGGAGCTGTG
ATGGAAGGCCAGCTCAACCAGGACGACTCCATCCTCAAGGTGGCTGTGAA
GACGATGAAGATTGCCATCTGCACGAGGTCAGAGCTGGAGGATTTCCTGA
GTGAAGCGGTCTGCATGAAGGAATTTGACCATCCCAACGTCATGAGGCTC
ATCGGTGTCTGTTTCCAGGGTTCTGAACGAGAGAGCTTCCCAGCACCTGT
GGTCATCTTACCTTTCATGAAACATGGAGACCTACACAGCTTCCTCCTCT
ATTCCCGGCTCGGGGACCAGCCAGTGTACCTGCCCACTCAGATGCTAGTG
AAGTTCATGGCAGACATCGCCAGTGGCATGGAGTATCTGAGTACCAAGAG
ATTCATACACCGGGACCTGGCGGCCAGGAACTGCATGCTGAATGAGAACA
TGTCCGTGTGTGTGGCGGACTTCGGGCTCTCCAAGAAGATCTACAATGGG
GACTACTACCGCCAGGGACGTATCGCCAAGATGCCAGTCAAGTGGATTGC
CATTGAGAGTCTAGCTGACCGTGTCTACACCAGCAAGAGCGATGTGTGGT
CCTTCGGGGTGACAATGTGGGAGATTGCCACAAGAGGCCAAACCCCATAT
CCGGGCGTGGAGAACAGCGAGATTTATGACTATCTGCGCCAGGGAAATCG
CCTGAAGCAGCCTGCGGACTGTCTGGATGGACTGTATGCCTTGATGTCGC
GGTGCTGGGAGCTAAATCCCCAGGACCGGCCAAGTTTTACAGAGCTGCGG
GAAGATTTGGAGAACACACTGAAGGCCTTGCCTCCTGCCCAGGAGCCTGA
CGAAATCCTCTATGTCAACATGGATGAGGGTGGAGGTTATCCTGAACCCC
CTGGAGCTGCAGGAGGAGCTGACCCCCCAACCCAGCCAGACCCTAAGGAT
TCCTGTAGCTGCCTCACTGCGGCTGAGGTCCATCCTGCTGGACGCTATGT
CCTCTGCCCTTCCACAACCCCTAGCCCCGCTCAGCCTGCTGATAGGGGCT
CCCCAGCAGCCCCAGGGCAGGAGGATGGTGCCTGA

Hence, preferably the AXL polypeptide or a biologically active variant or fragment thereof may be encoded by a nucleotide sequence substantially as set out in SEQ ID NO:2, or a variant or fragment thereof.

More preferably, the polypeptide comprises a soluble form of AXL. One embodiment of a suitable soluble form of AXL is provided herein as SEQ ID NO: 3, as follows:

[SEQ ID NO: 3]
eespfvgn pgnitgargl tgtlrcqlqv
ggeppevhwl rdgqilelad stqtqvplge deqddwivvs
qlritslqls dtgqyqclvf lghqtfvsqp gyvgleglpy
fleepedrtv aantpfnlsc gaggppepvd llwlgdavpl
atapghgpqr slhvpglnkt ssfsceahna kgvttsrtat
itvlpqqprn lhlvsrqpte levawtpgls giyplthctl
qavlsddgmg igagepdppe epltsgasvp phqlrlgslh
phtpyhirva ctssqgpssw thwlpvetpe gvplgppeni
satrngsqaf vhwqeprapl qgtllgyrla yqgqdtpevl
mdiglrqevt lelqgdgsys nitvcvaayt aagdgpwslp
vpleawrpgq aqpvhqlvke pstpafswp

Hence, in a more preferred embodiment, the AXL polypeptide comprises an amino acid sequence substantially as set out in SEQ ID NO: 3, or a variant or fragment thereof.

In one embodiment, the soluble form of AXL may be encoded by a nucleotide sequence, which is provided herein as SEQ ID NO: 4, as follows:

[SEQ ID NO: 4]
GAAG
AAAGTCCCTTCGTGGGCAACCCAGGGAATATCACAGGTGCCCGGGGACTC
ACGGGCACCCTTCGGTGTCAGCTCCAGGTTCAGGGAGAGCCCCCCGAGGT
ACATTGGCTTCGGGATGGACAGATCCTGGAGCTCGCGGACAGCACCCAGA
CCCAGGTGCCCCTGGGTGAGGATGAACAGGATGACTGGATAGTGGTCAGC
CAGCTCAGAATCACCTCCCTGCAGCTTTCCGACACGGGACAGTACCAGTG
TTTGGTGTTTCTGGGACATCAGACCTTCGTGTCCCAGCCTGGCTATGTTG
GGCTGGAGGGCTTGCCTTACTTCCTGGAGGAGCCCGAAGACAGGACTGTG
GCCGCCAACACCCCCTTCAACCTGAGCTGCCAAGCTCAGGGACCCCCAGA
GCCCGTGGACCTACTCTGGCTCCAGGATGCTGTCCCCCTGGCCACGGCTC
CAGGTCACGGCCCCCAGCGCAGCCTGCATGTTCCAGGGCTGAACAAGACA
TCCTCTTTCTCCTGCGAAGCCCATAACGCCAAGGGGGTCACCACATCCCG
CACAGCCACCATCACAGTGCTCCCCCAGCAGCCCCGTAACCTCCACCTGG
TCTCCCGCCAACCCACGGAGCTGGAGGTGGCTTGGACTCCAGGCCTGAGC
GGCATCTACCCCCTGACCCACTGCACCCTGCAGGCTGTGCTGTCAGACGA
TGGGATGGGCATCCAGGCGGGAGAACCAGACCCCCCAGAGGAGCCCCTCA
CCTCGCAAGCATCCGTGCCCCCCCATCAGCTTCGGCTAGGCAGCCTCCAT
CCTCACACCCCTTATCACATCCGCGTGGCATGCACCAGCAGCCAGGGCCC
CTCATCCTGGACCCACTGGCTTCCTGTGGAGACGCCGGAGGGAGTGCCCC
TGGGCCCCCCTGAGAACATTAGTGCTACGCGGAATGGGAGCCAGGCCTTC
GTGCATTGGCAAGAGCCCCGGGCGCCCCTGCAGGGTACCCTGTTAGGGTA
CCGGCTGGCGTATCAAGGCCAGGACACCCCAGAGGTGCTAATGGACATAG
GGCTAAGGCAAGAGGTGACCCTGGAGCTGCAGGGGGACGGGTCTGTGTCC
AATCTGACAGTGTGTGTGGCAGCCTACACTGCTGCTGGGGATGGACCCTG
GAGCCTCCCAGTACCCCTGGAGGCCTGGCGCCCAGGGCAAGCACAGCCAG
TCCACCAGCTGGTGAAGGAACCTTCAACTCCTGCCTTCTCGTGGCCC

Hence, in a more preferred embodiment, the AXL polypeptide may be encoded by a nucleotide sequence substantially as set out in SEQ ID NO: 4, or a variant or fragment thereof.

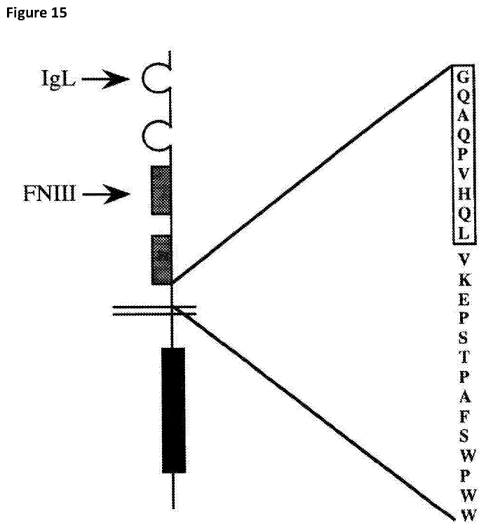

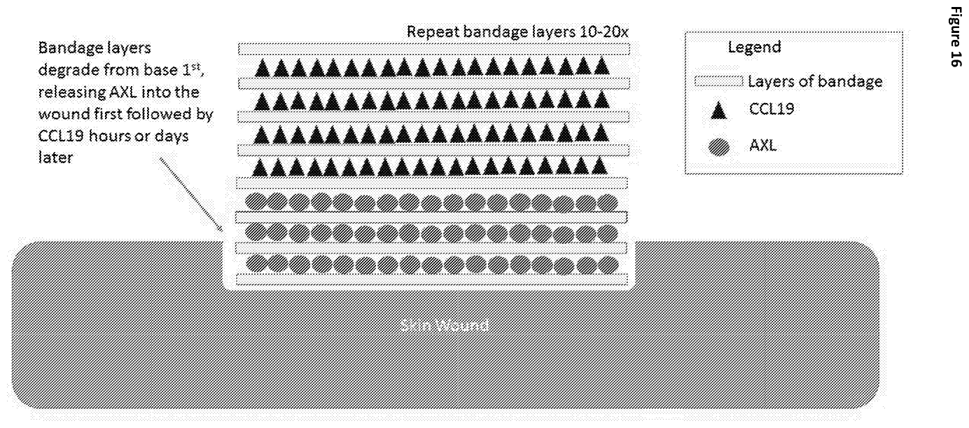

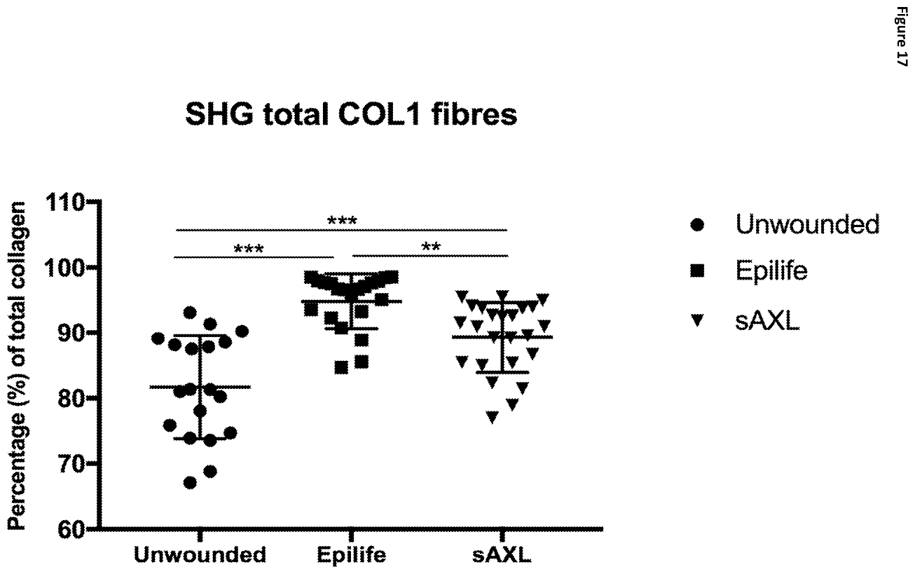

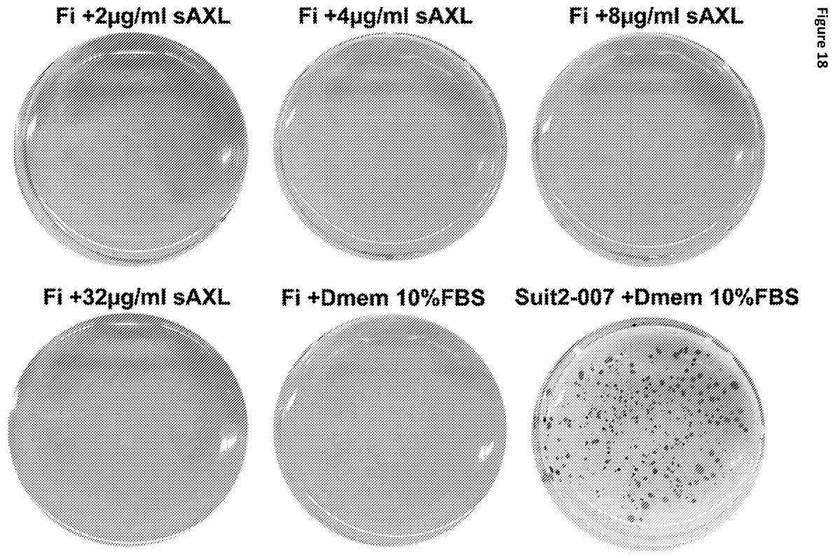

In one embodiment, the AXL variant polypeptide is a splice variant of AXL. Preferably, the splice variant lacks 9 amino acids (gqaqpvhql—SEQ ID No:14) at the C terminus of a fibronectin type III (FNIII) domain, as shown in FIG. 15. Hence, preferably the splice variant (NM_001699, NP_001690) is an 885 amino acid sequence as set out in SEQ ID No. 5.

[SEQ ID No: 5]
mawrcprmgr vplawclalc gwacmaprgt qaeespfvgn
pgnitgargl tgtlrcqlqv ggeppevhwl rdgqilelad
stqtqvplge deqddwivvs qlritslqls dtgqyqclvf
lghqtfvsqp gyvgleglpy fleepedrtv aantpfnlsc
gaggppepvd llwlgdavpl atapghgpqr slhvpglnkt
ssfsceahna kgvttsrtat itvlpqqprn lhlvsrqpte
levawtpgls giyplthctl qavlsddgmg igagepdppe
epltsgasvp phqlrlgslh phtpyhirva ctssqgpssw
thwlpvetpe gvplgppeni satrngsqaf vhwqeprapl
qgtllgyrla yqgqdtpevl mdiglrqevt lelqgdgsys
nitvcvaayt aagdgpwslp vpleawrpvk epstpafswp
wwyvllgavv aaacvlilal flvhrrkket rygevfeptv
ergelvvryr vrksysrrtt eatlnslgis eelkeklrdv
mvdrhkvalg ktlgegefga vmegglnqdd silkvavktm
kiaictrsel edflseavcm kefdhpnvmr ligvcfqgse
resfpapvvi lpfmkhgdlh sfllysrlgd qpvylptqml
vkfmadiasg meylstkrfi hrdlaarncm lnenmsvcva
dfglskkiyn gdyyrqgria kmpvkwiaie sladrvytsk
sdvwsfgvtm weiatrgqtp ypgvenseiy dylrqgnrlk
gpadoldgly almsrcweln pqdrpsftel redlentlka
lppagepdei lyvnmdeggg ypeppgaagg adpptqpdpk
dscscltaae vhpagryvlc psttpspaqp adrgspaapg
qedga

The term “active domain” in relation to AXL may relate to the minimal region of the AXL polypeptide that is capable of producing the wound healing, and prevention, reduction or inhibition of scar formation, effects of the invention. In particular, the active domain can relate to a region of AXL that interacts with a protein, preferably a receptor present on a keratinocyte.

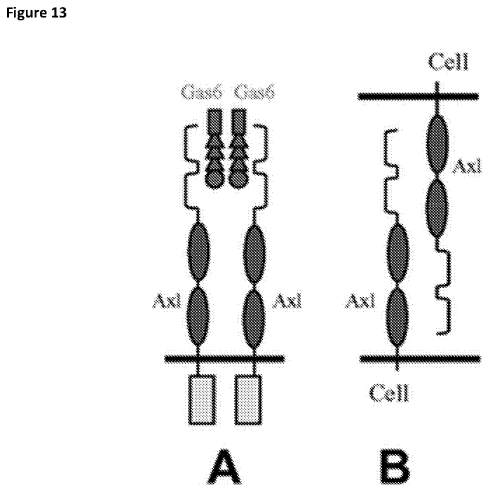

Although not wishing to be bound by hypothesis, the active domain may relate to a region of AXL that binds to the Gas6 receptor or alternatively binds to the extracellular domain of full length AXL (FIG. 13). The active domain may comprise at least one immunoglobulin (Ig) domain and/or at least one fibronectin type III (FNIII) domain. The active domain may comprise amino acid positions 37-428 of SEQ ID NO: 1. More preferably, the active domain comprises amino acid positions 37-124 (SEQ ID No: 6), 141-212 (SEQ ID No: 7), 224-322 (SEQ ID No: 8) and/or 325-428 (SEQ ID No: 9) of the amino acid as set out in SEQ ID NO: 1. Hence, most preferably, the active domain comprises amino acid sequences substantially as set out in (SEQ ID No: 6-9).

[SEQ ID No: 6]
fvgnpgnitgargltgtlrcqlqvggeppevhwlrdgqileladstqtqvp
lgedeqddwivvsqlritslqlsdtgqyqclvflghq
[SEQ ID No: 7]
Fleepedrtvaantpfnlscgaggppepvdllwlqdavplatapghgpqrs
lhvpglnktssfsceahnakg
[SEQ ID No: 8]
lpqqprnlhlvsrqptelevawtpglsgiyplthctlqavlsddgmgigag
epdppeepltsgasvpphqlrlgslhphtpyhirvactssqgpsswth
[SEQ ID No: 9]
pvetpegvplgppenisatrngsgafvhwgepraplqgtllgyrlayggqd
tpevlmdiglrgevtlelqgdgsysnltvcvaaytaagdgpwslpvpleaw
rp

The nucleotide sequences of each of these active domains are shown in SEQ ID No: 4, but the skilled person could readily design a nucleotide sequence to code for the active domains set out above. Thus, preferably the active domain is encoded by a variety of nucleotide sequences, to code for the sequences as set out in SEQ ID No: 6-b 9.

In another embodiment, the polypeptide is CCL19, or a biologically active variant or fragment thereof. In one embodiment, the human polypeptide CCL19 (Q5VZ75) is provided herein as SEQ ID NO: 10, as follows:

[SEQ ID NO: 10]
malllalsll vlwtspaptl sgtndaedcc lsvtqkpipg
yivrnfhyll ikdgcrvpav vfttlrgrql cappdqpwve
riiqrlqrts akaslalpgp vssl

Accordingly, preferably the CCL19 polypeptide comprises an amino acid sequence substantially as set out in SEQ ID NO: 10, or a biologically active variant or fragment thereof.

In one embodiment, CCL19 may be encoded by a nucleotide sequence (NM_006274) which is provided herein as SEQ ID NO: 11, as follows:

[SEQ ID NO: 11]
ATGGCCCTGCTACTGGCCCTCAGCCTGCTGGTTCTCTGGACTTCCCCAGC
CCCAACTCTGAGTGGCACCAATGATGCTGAAGACTGCTGCCTGTCTGTGA
CCCAGAAACCCATCCCTGGGTACATCGTGAGGAACTTCCACTACCTTCTC
ATCAAGGATGGCTGCAGGGTGCCTGCTGTAGTGTTCACCACACTGAGGGG
CCGCCAGCTCTGTGCACCCCCAGACCAGCCCTGGGTAGAACGCATCATCC
AGAGACTGCAGAGGACCTCAGCCAAGGCAAGCCTGGCCCTCCCTGGCCCT
GTCTCCTCCCTCTGA

Hence, preferably the CCL19 polypeptide or a biologically active variant or fragment thereof may be encoded by a nucleotide sequence substantially as set out in SEQ ID NO: 11, or a variant or fragment thereof.

In yet another embodiment, the polypeptide is BMP-6, or a biologically active variant or fragment thereof. In one embodiment, the human polypeptide BMP-6 (P22004) is provided herein as SEQ ID NO: 12 as follows:

[SEQ ID NO: 12]
mpglgrraqw lcwwwgllcs ccgppplrpp lpaaaaaaag
gqllgdggsp grteqpppsp qsssgflyrr lktgekremq
keilsvlglp hrprplhglq qpqppalrqg eeqqqqqqlp
rgepppgrlk saplfmldly nalsadnded gasegerqqs
wpheaasssq rrqpppgaah pinrksllap gsgsggaspl
tsaqdsafln dadmvmsfvn lveydkefsp rqrhhkefkf
nlsqipegev vtaaefriyk dcvmgsfknq tflisiyqvl
gehqhrdsdl flldtrvvwa seegwlefdi tatsnlwvvt
pqhnmglqls vvtrdgvhvh praaglvgrd gpydkqpfmv
affkvsevhv rttrsassrr rqqsrnrstq sqdvarvssa
sdynsselkt acrkhelyvs fqdlgwqdwi iapkgyaany
cdgecsfpin ahmnatnhai vqtivhlmnp eyvpkpccap
tklnaisvly fddnsnvilk kyrnmvvrac gch

Accordingly, preferably the BMP-6 polypeptide comprises an amino acid sequence substantially as set out in SEQ ID NO: 12, or a biologically active variant or fragment thereof.

In one embodiment, BMP-6 may be encoded by a nucleotide sequence (NM_001718) which is provided herein as SEQ ID NO: 13, as follows:

[SEQ ID NO: 13]
ATGCCGGGGCTGGGGCGGAGGGCGCAGTGGCTGTGCTGGTGGTGGGGGCT
GCTGTGCAGCTGCTGCGGGCCCCCGCCGCTGCGGCCGCCCTTGCCCGCTG
CCGCGGCCGCCGCCGCCGGGGGGCAGCTGCTGGGGGACGGCGGGAGCCCC
GGCCGCACGGAGCAGCCGCCGCCGTCGCCGCAGTCCTCCTCGGGCTTCCT
GTACCGGCGGCTCAAGACGCAGGAGAAGCGGGAGATGCAGAAGGAGATCT
TGTCGGTGCTGGGGCTCCCGCACCGGCCCCGGCCCCTGCACGGCCTCCAA
CAGCCGCAGCCCCCGGCGCTCCGGCAGCAGGAGGAGCAGCAGCAGCAGCA
GCAGCTGCCTCGCGGAGAGCCCCCTCCCGGGCGACTGAAGTCCGCGCCCC
TCTTCATGCTGGATCTGTACAACGCCCTGTCCGCCGACAACGACGAGGAC
GGGGCGTCGGAGGGGGAGAGGCAGCAGTCCTGGCCCCACGAAGCAGCCAG
CTCGTCCCAGCGTCGGCAGCCGCCCCCGGGCGCCGCGCACCCGCTCAACC
GCAAGAGCCTTCTGGCCCCCGGATCTGGCAGCGGCGGCGCGTCCCCACTG
ACCAGCGCGCAGGACAGCGCCTTCCTCAACGACGCGGACATGGTCATGAG
CTTTGTGAACCTGGTGGAGTACGACAAGGAGTTCTCCCCTCGTCAGCGAC
ACCACAAAGAGTTCAAGTTCAACTTATCCCAGATTCCTGAGGGTGAGGTG
GTGACGGCTGCAGAATTCCGCATCTACAAGGACTGTGTTATGGGGAGTTT
TAAAAACCAAACTTTTCTTATCAGCATTTATCAAGTCTTACAGGAGCATC
AGCACAGAGACTCTGACCTGTTTTTGTTGGACACCCGTGTAGTATGGGCC
TCAGAAGAAGGCTGGCTGGAATTTGACATCACGGCCACTAGCAATCTGTG
GGTTGTGACTCCACAGCATAACATGGGGCTTCAGCTGAGCGTGGTGACAA
GGGATGGAGTCCACGTCCACCCCCGAGCCGCAGGCCTGGTGGGCAGAGAC
GGCCCTTACGACAAGCAGCCCTTCATGGTGGCTTTCTTCAAAGTGAGTGA
GGTGCACGTGCGCACCACCAGGTCAGCCTCCAGCCGGCGCCGACAACAGA
GTCGTAATCGCTCTACCCAGTCCCAGGACGTGGCGCGGGTCTCCAGTGCT
TCAGATTACAACAGCAGTGAATTGAAAACAGCCTGCAGGAAGCATGAGCT
GTATGTGAGTTTCCAAGACCTGGGATGGCAGGACTGGATCATTGCACCCA
AGGGCTATGCTGCCAATTACTGTGATGGAGAATGCTCCTTCCCACTCAAC
GCACACATGAATGCAACCAACCACGCGATTGTGCAGACCTTGGTTCACCT
TATGAACCCCGAGTATGTCCCCAAACCGTGCTGTGCGCCAACTAAGCTAA
ATGCCATCTCGGTTCTTTACTTTGATGACAACTCCAATGTCATTCTGAAA
AAATACAGGAATATGGTTGTAAGAGCTTGTGGATGCCACTAA

Hence, preferably the BMP-6 polypeptide or a biologically active variant or fragment thereof may be encoded by a nucleotide sequence substantially as set out in SEQ ID NO: 13, or a variant or fragment thereof.

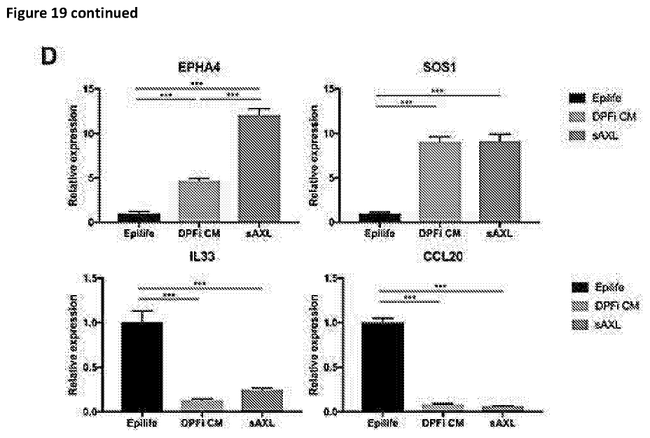

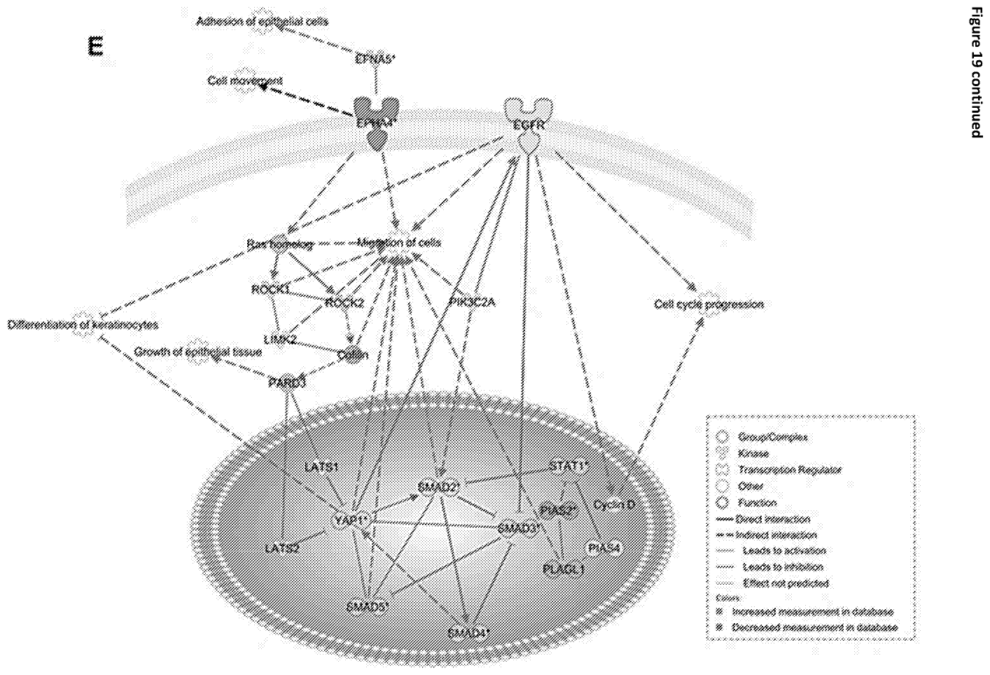

Advantageously, the polypeptides of the first aspect activate pathways that are associated with accelerated wound closure, as shown by the inventors in FIG. 19. Accordingly, in use, the polypeptides of the invention may activate the Hippo pathway, Ephrin pathway and/or the Epidermal Growth Factor (EGF) pathway. Preferably, in use, the polypeptides of the invention activate the Hippo pathway, Ephrin pathway and the Epidermal Growth Factor (EGF) pathway.

The skilled person would also consider the use of nucleic acids encoding polypeptides of the present invention, and vectors comprising nucleic acids encoding polypeptides of the present invention.

Accordingly, in a second aspect of the invention there is provided a vector comprising a nucleic acid sequence encoding a polypeptide sequence from a group consisting of AXL, CCL19 and BMP-6, or a biologically active variant or fragment thereof, for use in treating a wound.

The vector may comprise a nucleic acid encoding any polypeptide according to the first aspect of the invention.

The vector may for example be a plasmid, cosmid or phage and/or be a viral vector. Such recombinant vectors are highly useful in the delivery systems of the invention for transforming cells with the nucleic acid molecule. The nucleic acid sequence may preferably be a DNA sequence.

Preferably, the vector of the second aspect is recombinant. Recombinant vectors may also include other functional elements. For example, they may further comprise a variety of other functional elements including a suitable promoter for initiating transgene expression upon introduction of the vector in a host cell. For instance, the vector is preferably capable of autonomously replicating in the nucleus of the host cell. In this case, elements which induce or regulate DNA replication may be required in the recombinant vector. Alternatively, the recombinant vector may be designed such that it integrates into the genome of a host cell. In this case, DNA sequences which favour targeted integration (e.g. by homologous recombination) are envisaged. Suitable promoters may include the SV40 promoter, CMV, EFia, PGK, viral long terminal repeats, as well as inducible promoters, such as the Tetracycline inducible system, as examples. The cassette or vector may also comprise a terminator, such as the Beta globin, SV40 polyadenylation sequences or synthetic polyadenylation sequences. The recombinant vector may also comprise a promoter or regulator or enhancer to control expression of the nucleic acid as required. Tissue specific promoter/enhancer elements may be used to regulate expression of the nucleic acid in specific cell types, for example, epithelial cells. The promoter may be constitutive or inducible.

The vector may also comprise DNA coding for a gene that may be used as a selectable marker in the cloning process, i.e. to enable selection of cells that have been transfected or transformed, and to enable the selection of cells harbouring vectors incorporating heterologous DNA. For example, ampicillin, neomycin, puromycin or chloramphenicol resistance is envisaged. Alternatively, the selectable marker gene may be in a different vector to be used simultaneously with the vector containing the transgene. The cassette or vector may also comprise DNA involved with regulating expression of the transgene, or for targeting the expressed polypeptide to a certain part of the host cell.

Purified vector may be inserted directly into a host cell by suitable means, e.g. direct endocytotic uptake. The vector may be introduced directly into cells of a host subject (e.g. a eukaryotic or prokaryotic cell) by transfection, infection, electroporation, microinjection, cell fusion, protoplast fusion or ballistic bombardment. Alternatively, vectors of the invention may be introduced directly into a host cell using a particle gun.

The nucleic acid molecule may (but not necessarily) be one, which becomes incorporated in the DNA of cells of the subject being treated. Undifferentiated cells may be stably transformed leading to the production of genetically modified daughter cells (in which case regulation of expression in the subject may be required e.g. with specific transcription factors or gene activators). Alternatively, the delivery system may be designed to favour unstable or transient transformation of differentiated cells in the subject being treated. When this is the case, regulation of expression may be less important because expression of the DNA molecule will stop when the transformed cells die or stop expressing the protein (ideally when the required therapeutic effect has been achieved).

Alternatively, the delivery system may provide the nucleic acid molecule to the subject without it being incorporated in a vector. For instance, the nucleic acid molecule may be incorporated within a liposome or virus particle. Alternatively a “naked” nucleic acid molecule may be inserted into a subject's cells by a suitable means e.g. direct endocytotic uptake.

The nucleic acid molecule may be transferred to the cells of a subject to be treated by transfection, infection, microinjection, cell fusion, protoplast fusion or ballistic bombardment. For example, transfer may be by ballistic transfection with coated gold particles, liposomes containing the nucleic acid molecule, viral vectors (e.g. adenovirus) and means of providing direct nucleic acid uptake (e.g. endocytosis) by application of the nucleic acid molecule directly.

The wound treatment according to the invention preferably comprises re-epithelisation of epithelial tissue. Re-epithelialisation is defined as the restoration of an intact epithelium through migration of epithelial cells to close a wound. Epithelia coat all surfaces of the body, both inside and out. Therefore, the treatment comprises re-epithelisation and can be used in any epithelial wound, i.e. internal or external of the body.

Preferably, the rate of wound healing is increased and/or scar formation is prevented, reduced or inhibited.

While the skilled person would understand that the term “treating a wound” encompasses prevention, reduction or inhibition of scar formation, the invention also extends to the use of the polypeptides, nucleic acids or vectors of invention in preventing, reducing or inhibiting scar formation in a wound that has already closed and/or has already been treated. Thus, the invention extends to the polypeptides, nucleic acids or vectors of the invention, for use in preventing, reducing or inhibiting scar formation per se.

In a third aspect of the invention, there is provided a method of treating a wound, the method comprising administering, to a subject in need thereof, a therapeutic amount of a polypeptide selected from the group consisting of AXL, CCL19 and BMP-6, or a biologically active variant or fragment thereof.

Preferably, the method comprises administering a therapeutic amount of AXL, or a biologically active variant or fragment thereof in a temporal manner, preferably wherein the biologically active variant or fragment thereof comprises the active domain of AXL. The method may further comprise administration of CCL19 and/or BMP-6, or a biologically active variant or fragment thereof. Preferably, the method comprises administering a therapeutic amount of a polypeptide selected from the group consisting of AXL, or a biologically active variant or fragment thereof, and CCL19, or a biologically active variant or fragment thereof.

In another embodiment, the method comprises administering a therapeutic amount of CCL19, or a biologically active variant or fragment thereof.

In another embodiment, the method comprises administering a therapeutic amount of BMP-6, or a biologically active variant or fragment thereof.

The method may comprise administration of a vector comprising a nucleic acid sequence encoding a polypeptide selected from a group consisting of AXL, CCL19 and BMP-6, or a biologically active variant or fragment thereof, for treating the wound.

Rate of Wound Healing

Preferably, the polypeptides of the invention increase the rate of wound healing. The rate of wound healing may relate to the absolute area healed per day, percentage of initial area healed per day and advance of the wound margin towards the wound centre per day, time to complete wound closure, or any other method known in the art, including those described herein. An increase in the rate of wound healing refers to that achieved compared to the level of healing occurring on healing of a control-treated or untreated wound.

Prevention, Reduction or Inhibition of Scarring

Preferably, the polypeptides of the invention result in the prevention, reduction or inhibition of scarring. The inventors believe that this is an important aspect of the invention.

Thus, in a further aspect of the invention, there is provided a polypeptide selected from the group consisting of AXL, CCL19 and BMP-6, or a biologically active variant or fragment thereof, for use in preventing, reducing or inhibiting scarring.

The prevention, reduction or inhibition of scarring within the context of the present invention should be understood to encompass any degree of prevention, reduction or inhibition in scarring achieved on healing of a treated wound, as compared to the level of scarring occurring on healing of a control-treated or untreated wound. Throughout the specification references to “prevention”, “reduction” or “inhibition” of scarring are generally to be taken, except where the context requires otherwise, to represent substantially equivalent activities, involving equivalent mechanisms mediated by polypeptides of the present invention.

Assessment of Scarring

The extent of inhibition of scarring that may be required in order to achieve a therapeutic effect will be apparent to, and may readily be determined by, a clinician responsible for the care of the patient. The clinician may undertake a suitable determination of the extent of inhibition of scarring that has been achieved, in order to assess whether or not a therapeutic effect has been achieved, or is being achieved. Such an assessment may, but need not necessarily, be made with reference to suggested methods of measurement described herein.

The extent to which inhibition of scarring after wound closure is achieved may be assessed with reference to the effects that such an active agent may achieve in human patients treated with the methods or medicaments of the invention. Alternatively, inhibition of scarring that may be achieved may be assessed with reference to experimental investigations using suitable in vitro or in vivo models. The use of experimental models to investigate inhibition of scarring may be particularly preferred in assessing the therapeutic effectiveness of the polypeptides of the present invention, or in establishing therapeutically effective amounts of such polypeptides.

Animal models of wound healing and scar formation represent preferred experimental models for in vivo assessment of the extent of scar inhibition that may be achieved using the medicaments or methods of the invention. Examples of such models are described below for illustrative purposes. The models of scarring and methods for assessing scarring described herein may be used to determine therapeutically effective polypeptides.

Inhibition of scarring, using the medicaments and methods of the invention, can be effected at any body site and in any tissue or organ so far investigated. For illustrative purposes the scar inhibitory activity of medicaments and methods of the invention will primarily be described with reference to inhibition of scarring that may be brought about in the skin (the body's largest organ). However, the skilled person will immediately appreciate that many of the factors that are relevant when considering inhibition of scarring in the skin are also relevant to inhibition of scarring in other organs or tissues. Accordingly the skilled person will recognise that, except for where the context requires otherwise, the parameters and assessments considered below in respect of scars of the skin may also be applicable to scarring in tissues other than the skin. The skilled person will recognise that the above is equally applicable in the context of re-epithelisation and the rate of wound healing and is not limited to the assessment of scarring.

In the skin, treatment of wounds may improve the macroscopic and microscopic appearance of scars which arise when these wounds close; macroscopically the scars may be less visible and blend with the surrounding skin, microscopically the collagen fibres within the scar may have morphology and anisotropic organisation that is more similar to those in the surrounding skin.

The inhibition of scarring achieved using methods and medicaments of the invention may be assessed and/or measured with reference to either the microscopic or macroscopic appearance of a scar generated by treatment of a wound to promote closure as compared to the appearance of a scar formed by closure of a wound with no polypeptide treatment. Inhibition of scarring may also suitably be assessed with reference to both macroscopic and microscopic appearance of a treated scar.

In considering the macroscopic appearance of a scar resulting from a treated wound, the extent of scarring, and hence the magnitude of any inhibition of scarring achieved, may be assessed with reference to any of a number of parameters. Most preferably, holistic assessment of the scar by means of assessment of macroscopic photographs by an independent expert panel, by means of an independent lay panel or clinically by means of a macroscopic assessment by a clinician of the patients themselves. Assessments are captured by means of a VAS (visual analogue scale) or a categorical scale.

Macroscopic characteristics of a scar which can be assessed objectively include: i) Colour of the scar. Scars may typically be hypopigmented or hyperpigmented with regard to the surrounding skin. Inhibition of scarring may be demonstrated when the pigmentation of a treated scar more closely approximates that of unscarred skin than does the pigmentation of an untreated scar. Similarly, scars may be redder than the surrounding skin. In this case inhibition of scarring may be demonstrated when the redness of a treated scar fades earlier, or more completely, or to resemble more closely the appearance of the surrounding skin, compared to an untreated scar. There are a number of non-invasive colorimetric devices which are able to provide data with respect to pigmentation of scars and unscarred skin, as well as redness of the skin (which may be an indicator of the degree of vascularity present in the scar or skin). Examples of such devices include the X-rite SP-62 spectrophotometer, Minolta Chronometer CR-200/300; Labscan 600; Dr. Lange Micro Colour; Derma Spectrometer; laser-Doppler flow meter; and Spectrophotometric intracutaneous Analysis (SIA) scope. ii) Height of the scar. Scars may typically be either raised or depressed as compared to the surrounding skin. Inhibition of scarring may be demonstrated when the height of a treated scar more closely approximates that of unscarred skin (i.e. is neither raised nor depressed) than does the height of an untreated scar. Height of the scar can be measured directly on a patient by means of profilometry, or indirectly, by profilometry of moulds taken from a scar. iii) Surface texture of the scar. Scars may have surfaces that are relatively smoother than the surrounding skin (giving rise to a scar with a “shiny” appearance) or that are rougher than the surrounding skin. Inhibition of scarring may be demonstrated when the surface texture of a treated scar more closely approximates that of unscarred skin than does the surface texture of an untreated scar. Surface texture can be measured directly on a patient by means of profilometry, or indirectly by profilometry of moulds taken from a scar. iv) Stiffness of the scar. The abnormal composition and structure of scars means that they are normally stiffer than the undamaged skin surrounding the scar. In this case, inhibition of scarring may be demonstrated when the stiffness of a treated scar more closely approximates that of unscarred skin than does the stiffness of an untreated scar.

A treated scar will preferably exhibit inhibition of scarring as assessed with reference to at least one of the parameters for macroscopic assessment set out in the present specification. More preferably a treated scar may demonstrate inhibited scarring with reference to at least two parameters, even more preferably at least three parameters, and most preferably at least four of these parameters (for example, all four of the parameters set out above). The parameters described above may be used in the development of a visual analogue scale (VAS) for the macroscopic assessment of scarring. Details regarding implementation of VASs are described below. Microscopic assessment may also provide a suitable means by which the quality of treated and untreated or control scars may be compared. Microscopic assessment of scar quality may typically be carried out using histological sections of scars. Suitable parameters for the microscopic assessment of scars may include: i) Thickness of extracellular matrix (ECM) fibres. Inhibition of scarring may be demonstrated when the thickness of ECM fibres in a treated scar more closely approximates the thickness of ECM fibres found in unscarred skin than does the thickness of fibres found in an untreated scar. ii) Orientation of ECM fibres. ECM fibres found in scars tend to exhibit a greater degree of alignment with one another than do those found in unscarred skin (which have a random orientation frequently referred to as “basket weave”). Accordingly, inhibition of scarring may be demonstrated when the orientation of ECM fibres in a treated scar more closely approximates the orientation of ECM fibres found in unscarred skin than does the orientation of such fibres found in an untreated scar. iii) ECM composition of the scar. The composition of ECM molecules present in scars shows differences from that found in normal skin, with a reduction in the amount of elastin present in ECM of scars. Thus inhibition of scarring may be demonstrated when the composition of ECM fibres in the dermis of a treated scar more closely approximates the composition of such fibres found in unscarred skin than does the composition found in an untreated scar. iv) Cellularity of the scar. Scars tend to contain relatively fewer cells than does unscarred skin. It will therefore be appreciated that inhibition of scarring may be demonstrated when the cellularity of a treated scar more closely approximates the cellularity of unscarred skin than does the cellularity of an untreated scar. v) Appendages. Scars do not contain adnexal structures such as glands or hair follicles. The presence of these in the treated skin will indicate that functional tissue regeneration rather than scar formation has occurred.

Other features that may be taken into account in assessing the microscopic quality of scars include elevation or depression of the scar relative to the surrounding unscarred skin, and the prominence or visibility of the scar at the interface with the unscarred skin.

The parameters described above may be used in generating a VAS for the microscopic assessment of scarring. Such a VAS may consider collagen organisation and abundance in the papillary dermis and the reticular dermis may also provide a useful index of scar quality. Inhibition of scarring may be indicated when the quality of a treated scar is closer to that of unscarred skin than is the quality of an untreated or control scar. It is surprising to note that the overall appearance of scars, such as those of the skin, is little influenced by the epidermal covering of the scar, even though this is the part of the scar that is seen by the observer. Instead, the inventors find that the properties of the connective tissue (such as that making up the dermis, or neo-dermis) present within the scar have greater impact on the perception of extent of scarring, as well as on the function of the scarred tissue. Accordingly assessments of criteria associated with the connective tissues such as the dermis, rather than epidermis, may prove to be the most useful in determining inhibition of scarring.

The thickness of ECM fibres and orientation of ECM fibres may be favoured parameters, for assessing inhibition of scarring. A treated scar may preferably have improved ECM orientation (i.e. orientation that is more similar to unscarred skin than is the orientation in an untreated scar).

A treated scar will preferably demonstrate inhibition of scarring as assessed with reference to at least one of the parameters for microscopic assessment set out above. More preferably a treated scar may demonstrate inhibition of scarring with reference to at least two of the parameters, even more preferably at least three of the parameters, even more preferably at least four of the parameters, and most preferably all five of these parameters.

It will be appreciated that inhibition of scarring achieved using the medicaments or methods of the invention may be indicated by improvement of one or more suitable parameters combined from different assessment schemes (e.g. inhibition as assessed with reference to at least one parameter used in macroscopic assessment and at least one parameter used in microscopic assessment).

Further examples of suitable parameters for the clinical measurement and assessment of scars may be selected based upon a variety of measures or assessments including those described by Duncan et al. (2006), Beausang et al. (1998) and van Zuijlen et al (2002). Except for where the context requires otherwise, many of the following parameters may be applied to macroscopic and/or microscopic assessment of scarring. Examples of Suitable parameters for assessment of scars in the skin may include:

1. Assessment with regard to Visual Analogue Scale (VAS) scar score.

Prevention, reduction or inhibition of scarring may be demonstrated by a reduction in the VAS score of a treated scar when compared to a control scar. A suitable VAS for use in the assessment of scars may be based upon the method described by Duncan et al. (2006) or by Beausang et al. (1998). This is typically a 10 cm line in which 0 cm is considered an imperceptible scar and 10 cm a very poor hypertrophic scar.

2. Assessment with regard to a categorical scale.

Prevention, reduction or inhibition of scarring may be determined by allocating scars to different categories based on either textual descriptions e.g. “barely noticeable”, “blends well with normal skin”, “distinct from normal skin”, etc., by comparing a treated scar and a an untreated or control scar, noting any differences between these, and allocating the differences to selected categories (suitable examples of which may be “mild difference”, “moderate difference”, “major difference”, etc.). Assessment of this sort may be performed by the patient, by an investigator, by an independent panel, or by a clinician, and may be performed either directly on the patient or on photographs or moulds taken from the patient. Inhibition of scarring may be demonstrated when an assessment indicates that treated scars are generally allocated to more favourable categories than are untreated or control scars.

3. Scar height, scar width, scar perimeter, scar area or scar volume.

The height and width of scars can be measured directly upon the subject, for example by use of manual measuring devices such as callipers, or automatically with the use of profilometers. Scar width, perimeter and area may be measured either directly on the subject, by image analysis of photographs of the scar, by analysis of silicone mould impressions of the scar, or by analysis of positive casts made from such impressions. The skilled person will also be aware of further non-invasive methods and devices that can be used to investigate suitable parameters, including silicone moulding, ultrasound, optical three-dimensional profilimetry and high resolution Magnetic Resonance Imaging. Inhibition of scarring may be demonstrated by a reduction in the height, width, area, perimeter or volume, or any combination thereof, of a treated scar as compared to an untreated scar.

4. Scar distortion and mechanical performance

Scar distortion may be assessed by visual comparison of a scar and unscarred skin. A suitable comparison may categorise a selected scar as causing no distortion, mild distortion, moderate distortion or severe distortion.

The mechanical performance of scars can be assessed using a number of non-invasive methods and devices based upon suction, pressure, torsion, tension and acoustics. Suitable examples of devices capable of use in assessing mechanical performance of scars include Indentometer, Cutometer, Reviscometer, Visco-elastic skin analysis, Dermaflex, Durometer, Dermal Torque Meter and Elastometer. Inhibition of scarring may be demonstrated by a reduction in distortion caused by treated scars as compared to that caused by untreated scars. It will also be appreciated that inhibition of scarring may be demonstrated by the mechanical performance of unscarred skin being more similar to that of treated scars than of untreated scars.

Photographic Assessments Independent Lay Panel

Photographic assessment of treated and untreated scars may be performed by an independent lay panel of assessors using standardised and calibrated photographs of the scars. The scars may be assessed by an independent lay panel to provide categorical ranking data (e.g. that a given treated scar is “better”, “worse” or “no different” when compared to an untreated scar) and quantitative data using a Visual Analogue Scale (VAS) based upon the method described by Duncan et al. (2006) and Beausang et al. (1998).

Expert Panel

Photographic assessment of treated and untreated scars may alternatively or additionally be performed by a panel of expert assessors using standardised and calibrated photographs of the scars to be assessed, and/or positive casts of silicone moulds. The panel of experts may preferably consist of individuals skilled in the art, suitable examples of which include plastic surgeons, dermatologists or scientists having relevant technical backgrounds.

Clinical Assessment

A clinician, or an independent panel of clinicians may assess the scar(s) on a patient using any of the forgoing parameters e.g. VAS, colour, categorical scales, etc. A suitable clinician may be a clinician responsible for care of a patient, or may be a clinician investigating efficacy of therapies for inhibition of scarring.

Patient Assessment

A patient may assess their own scars and/or compare scars by means of a structured questionnaire. A suitable questionnaire may measure parameters such as: the patient's satisfaction with their scar; how well the scar blends with the unscarred skin; as well as the effect of the scar on their daily life (suitable questions may consider whether the patient uses clothes to hide the scar, or otherwise avoids exposing it) and/or scar symptoms (examples of which may include itch, pain or paresthesia). Inhibition of scarring may be indicated by the treated scar receiving a more positive rating from the patient, and/or causing the patient fewer problems, and/or causing fewer or less scar symptoms, and/or an increase in patient satisfaction compared to an untreated scar. In addition to categorical data, quantitative data (preferably relating to the above parameters) can be generated using image analysis in combination with suitable visualisation techniques. Examples of suitable visualisation techniques that may be employed in assessing scar quality are specific histological stains or irnmuno-labelling, wherein the degree of staining or labelling present may be quantitatively determined by image analysis.

Quantitative data may be usefully and readily produced in relation to the following parameters:

1. Scar width, height, elevation, volume and area.

2. Collagen organisation, collagen fibre thickness, collagen fibre density.

3. Number and orientation of fibroblasts.

4. Quantity and orientation of other ECM molecules e.g. elastin, fibronectin

Prevention, reduction or inhibition of scarring may be demonstrated by a change in any of the parameters considered above such that a treated scar more closely resembles unscarred skin than does a control or untreated scar (or other suitable comparator). The assessments and parameters discussed above are suitable for assessment of the effects of a polypeptide, on scar formation, as compared to control, placebo or standard care treatment in animals or humans. It will be appreciated that these assessments and parameters may be utilised in determining a therapeutically effective polypeptide that may be used for scar prevention, reduction or inhibition; and in determining therapeutically effective amounts of polypeptides of the invention, such as AXL. Appropriate statistical tests may be used to analyse data sets generated from different treatments in order to investigate the significance of results.

Other parameters that may be used in the assessment of scarring in organs other than the skin may be determined with reference to the organ in question. For example, corneal scarring may be assessed by measuring the opacity, or transmitting/refractory properties, of the cornea and measurement of corneal curvature. Such assessments may, for example, be made using in vivo confocal microscopy and/or specular microscopy or corneal topography.

Successful inhibition of scarring in tendons or ligaments may be indicated by restoration of function of tissues treated with the medicaments or methods of the invention. Suitable indicators of function may include the ability of the tendon or ligament to bear weight, stretch, flex, etc. Such assessments may, for example, be made using electrophysiological reflex examination, surface electromyography, ultrasonography, ultrasound/MRI scan, and self reported symptom and pain questionnaires.

The extent of scarring occurring in blood vessels can be measured directly e.g. using ultrasound, or indirectly by means of blood flow. Inhibition of scarring achieved using the medicaments or methods of the invention may lead to a reduction in narrowing of the blood vessel lumen and allow a more normal blood flow.

Wound Sites

The wound may be present at any body site, and in any tissue or organ, where a wound may occur. The skin represents the preferred site at which the rate of wound healing is increased and/or scar formation is prevented, reduced or inhibited. The inventors believe that the polypeptides of the present invention may beneficially increase wound healing and reduce scar formation in all types of epithelial wounds. Examples of specific wounds in which the effects of the invention may be seen include wounds selected from the group consisting of wounds of the skin (such as burns, incision wounds, pressure ulcers), the lungs, the eye (including the inhibition of scarring resulting from eye surgery such as LASIK surgery, LASEK surgery, PRK surgery, glaucoma filtration surgery, cataract surgery, or surgery in which the lens capsule may be subject to scarring) such as those giving rise to corneal cicatrisation; wounds subject to capsular contraction (which is common surrounding breast implants); wounds of the oral cavity, including the lips and palate (for example, to inhibit scarring resulting from treatment of cleft lip or palate or to promote closure or oral ulcers); wounds of the internal organs such as the digestive tissues and reproductive tissues; wounds of body cavities such as the abdominal cavity, pelvic cavity and thoracic cavity (where inhibition of scarring may reduce the number of incidences of adhesion formation and/or the size of adhesions formed); and surgical wounds (in particular wounds associated with cosmetic procedures, such as scar revision or isolation of strip grafts for hair transplant surgery). It is particularly preferred that the polypeptides of the present invention be used to increase the rate of re-epithelisation, wound healing and/or prevent, reduce or inhibit scarring associated with wounds of the skin.

Incisional wounds are a preferred group of wounds resulting in scarring which may be inhibited by the polypeptides of the invention. Surgical incisional wounds may constitute a particularly preferred group of wounds in respect of which wound healing and/or scarring may be inhibited utilising the medicaments and methods of the invention.

Polypeptides of the present invention may be used to heal wounds and/or inhibit scarring associated with plastic or cosmetic surgery. Since a large number of plastic or cosmetic surgeries consist of elective surgical procedures it is readily possible to administer a polypeptide of the present invention, prior to surgery, and/or around the time of closure of the wound (for instance, before or after the application of sutures), and this use represents a particularly preferred embodiment of the invention. In surgical procedures in general, a preferred route by which a polypeptide of the present invention may be administered is via localised injection (such as intradermal injection). Such injections may form raised blebs, which may then be incised as part of the surgical procedure, or alternatively the bleb may be raised by injecting the wound margins after the wound has been closed e.g. by sutures. Alternatively, the polypeptide may be administered in a cream formulation or in a bandage, or may be coated on the sutures used for incision closure.

Scar revisions are surgical procedures in which existing scars are “revised” (for example through excision or realignment) in order to reduce the cosmetic and/or mechanical disruption caused by the existing scar. Probably the best known of these is “Z-plasty” in which two V-shaped flaps of skin are transposed to allow rotation of a line of tension. The use of the polypeptides of the invention in procedures associated with scar revision represents a preferred use in accordance with the present invention.

It is recognised that wounds resulting from burns injuries (which for the purposes of the present invention may be taken to encompass exposure to heated gasses or solids, as well as scalding injuries involving hot liquids; “freezer burn” injuries caused by exposure to extreme low temperatures; radiation burns; and chemical burns, such as those caused by caustic agents) may extend over great areas of an individual so afflicted. Accordingly, burns may give rise to scar formation covering a large proportion of a patient's body. This great extent of coverage increases the risk that the scar formed will cover areas of elevated cosmetic importance (such as the face, neck, arms or hands) or of mechanical importance (particularly the regions covering or surrounding joints). Burns injuries caused by hot liquids are frequently suffered by children (for example as a result of upsetting pans, kettles or the like) and, due to the relatively smaller body size of children, are particularly likely to cause extensive damage over a high proportion of the body area. Thus there is an elevated risk of both cosmetic and mechanical impairment associated with scarring after burns. After large burns, skin grafts are used as a treatment. This invention can be used in combination with a skin graft, to promote migration of epithelial cells from the graft to the uncovered wound, to quickly establish a barrier in non-grafted areas of skin.

It will be appreciated that the polypeptides according to the invention may be used in a medicament, which may be used as a monotherapy (i.e. use of the polypeptides according to the first aspect), for treating wound, in particular to increasing the rate of wound healing and/or preventing, reducing or inhibiting scarring. Alternatively, the polypeptides according to the invention may be used as an adjunct to, or in combination with, known therapies for treating a wound, in particular to increasing the rate of wound healing and/or preventing, reducing or inhibiting scarring.

The polypeptide according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

The polypeptides according to the invention may also be incorporated within a slow- or delayed-release device such as a layer-by-layer assembled bandage. Such devices may, for example, be inserted on or under the skin, and the medicament may be released over weeks or even months. The device may be located at least adjacent to the treatment site. Such devices may be particularly advantageous when long-term treatment with the polypeptide is required and which would normally require frequent administration (e.g. at least daily injection).

It will be appreciated that the amount of the polypeptides that is required is determined by its biological activity and bioavailability, which in turn depends on the mode of administration, the physiochemical properties of the polypeptide and whether it is being used as a monotherapy or in a combined therapy. The frequency of administration will also be influenced by the half-life of the cyclic polypeptide within the subject being treated. Optimal dosages to be administered may be determined by those skilled in the art, and will vary with the particular polypeptide in use, the strength of the pharmaceutical composition, the mode of administration, and the advancement or stage of the disorder. Additional factors depending on the particular subject being treated will result in a need to adjust dosages, including subject age, weight, gender, diet, and time of administration.

Generally, a daily dose of between 0.001 μg/kg of body weight and 10 mg/kg of body weight, or between 0.01 μg/kg of body weight and 1 mg/kg of body weight, of the construct or vector according to the invention may be used for treating a wound, in particular to increasing the rate of wound healing and/or preventing, reducing or inhibiting scarring, depending upon the polypeptide used. Preferably, AXL polypeptides of the invention are applied at a concentration of between 1-3 μg/ml, more preferably at a concentration of about 2 μg/ml. CCL19 polypeptides of the invention are applied at a concentration of between 0.1-2.5 ng/ml, more preferably at 0.5 ng/ml while BMP6 is applied at 0.1-0.6 μg/ml, and more preferably at 0.03 μg/ml.

The polypeptides may be administered before, during or after onset of the injury causing the wound. Daily doses may be given as a single administration (e.g. a topical cream or spray). Alternatively, the polypeptide may require administration twice or more times during a day. As an example, the polypeptide may be administered as two (or more depending upon the severity of the disorder being treated) daily doses of between 0.07 μg and 700 mg (i.e. assuming a body weight of 70 kg). A patient receiving treatment may administer a first dose upon waking and then a second dose in the evening (if on a two dose regime) or at 3- or 4-hourly intervals thereafter. Alternatively, a slow release device may be used to provide optimal doses of the polypeptide according to the invention to a patient without the need to administer repeated doses.

Known procedures, such as those conventionally employed by the pharmaceutical industry (e.g. in vivo experimentation, clinical trials, etc.), may be used to form specific formulations of the polypeptide according to the invention and precise therapeutic regimes (such as daily doses of the agents and the frequency of administration). The inventors believe that they are the first to show that AXL, CCL19 and/or BMP-6 are surprisingly effective in treating a wound, in particular in increasing the rate of wound healing and/or preventing, reducing or inhibiting scarring.

According to a fourth aspect of the invention, there is provided a wound treatment pharmaceutical composition comprising a polypeptide selected from the group consisting of AXL, CCL19 and BMP-6, or a biologically active variant or fragment thereof, or a vector comprising a nucleic acid sequence encoding a polypeptide sequence from a group consisting of AXL, CCL19 and BMP-6, or a biologically active variant or fragment thereof, and a pharmaceutically acceptable vehicle.

The polypeptide may be as defined in the first aspect and the vector may be as defined in the second aspect.

According to a fifth aspect, there is provided a method of preparing the wound treatment pharmaceutical composition according to the fourth aspect, the method comprising contacting a polypeptide selected from the group consisting of AXL, CCL19 and BMP-6, or a biologically active variant or fragment thereof, or a vector comprising a nucleic acid sequence encoding a polypeptide sequence from a group consisting of AXL, CCL19 and BMP-6, or a biologically active variant or fragment thereof, with a pharmaceutically acceptable vehicle.

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the polypeptide or the pharmaceutical composition is any amount which, when administered to a subject, is the amount of the aforementioned that is needed to treat a wound, in particular to increase the rate of wound healing and/or prevent, reduce or inhibit scarring.

For example, the therapeutically effective amount of the polypeptide or the pharmaceutical composition used may be from about 0.01 mg to about 800 mg, and preferably from about 0.01 mg to about 500 mg. It is preferred that the amount of the polypeptide or the pharmaceutical composition is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The polypeptide according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

The polypeptides and the pharmaceutical composition of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The polypeptide or the pharmaceutical composition according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions. In the situation in which it is desired to administer a polypeptide of the present invention by means of oral ingestion, it will be appreciated that the chosen agonist will preferably be one having an elevated degree of resistance to degradation. For example, the chosen agonist may be protected (using the techniques well known to those skilled in the art) so that its rate of degradation in the digestive tract is reduced.

Medicaments comprising a polypeptide of the present invention that are for use in treating wounds in the lungs or other respiratory tissues may be formulated for inhalation.

Any suitable route capable of achieving the desired effect of the invention can be used to administer a therapeutically effective amount of a polypeptide of the present invention. However, it may generally be preferred that the polypeptide of the invention is provided to a tissue by local administration.

Suitable methods by which such local administration may be achieved will depend on the identity of the tissue or organ in question. The selection of preferred routes of administration may also depend on whether or not a tissue or organ to be treated is permeable to the chosen medicament. Suitable routes of administration may be selected from the group consisting of: injections; application of sprays, ointments, gels or creams; inhalation of medicaments; release from biomaterials or other solid medicaments including sutures or wound dressings.

Suitable delivery systems may include particulate systems, scaffolds or hydrogels. Particulate particles include micro particles or nanoparticles. Such particulate particles may be lipid based or polymer based. Preferably, polymer based particles are biodegradable. Scaffolds may include those biomaterials derived from native ECM, such as HA, collagen, and chitosan. Scaffolds may also comprise biomimetic materials fabricated to mimic ECM, including micro/nanofibers scaffolds produced by electrospinning. The polypeptides of the invention may preferably be provided in the form of one of more dosage units providing a therapeutically effective amount (or a known fraction or multiple of a therapeutically effective amount) of polypeptides of the invention. Methods of preparing such dosage units will be well known to the skilled person; for example see Remington's Pharmaceutical Sciences 18th Ed. (1990).

Suitable polypeptides may be provided on a sterile dressing or patch, which may be used to cover a wound where a wound is to be treated.

A polypeptide of the invention may be released from a device or implant, or may be used to coat such a device, e.g. a stent, or a controlled release device, or a wound dressing, or sutures for use in wound closure.

It will be appreciated that the vehicle of a composition comprising a polypeptide of the invention should be one that is well tolerated by the patient and allows release of the polypeptide to the wound to be treated. Such a vehicle is preferably relatively “mild” i.e. non-inflammatory, biodegradeable, bioresolveable, or bioresorbable.

A dose of a composition comprising a polypeptide of the present invention may preferably be sufficient to provide a therapeutically effective amount of a suitable agonist in a single administration. However, it will be appreciated that each dose need not in itself provide a therapeutically effective amount of a polypeptide of the present invention, but that a therapeutically effective amount may instead be built up through repeated administration of suitable doses.

In a further embodiment, polypeptide of the invention may be formulated as a part of a pharmaceutically acceptable trans-epidermal delivery system, e.g. a patch/dressing. A solid vehicle can include one or more substances that may also act as flavouring agents, lubricants, solubilizers, suspending agents, fillers, glidants, compression aids, binders or tablet-disintegrating agents; it can also comprise an encapsulating material.

Medicaments in accordance with the invention for use in treating wounds in the body cavities e.g. abdomen or pelvis, may be formulated as an irrigation fluid, lavage, gel or instillate.

Polypeptides for use in the medicaments or methods of the invention may be incorporated in a biomaterial, from which it may be released to treat a wound, in particular.

Biomaterials incorporating polypeptide of the present invention are suitable for use in many contexts, and at many body sites but may be of particular utility in providing a suitable polypeptides of the invention to the eye (for example after retina surgery or glaucoma filtration surgery), or to sites where it is wished to inhibit restenosis or adhesions. The inventors believe that biomaterials incorporating polypeptides of the invention may be used in the manufacture of sutures, and such sutures represent a preferred embodiment of a medicament of the invention.

Accordingly, in a sixth aspect of the invention, there is provided a device comprising a polypeptide selected from the group consisting of AXL, CCL19 and BMP-6, or a biologically active variant or fragment thereof, wherein the device is configured for the controlled spatio-temporal delivery of the polypeptide.

Preferably, the controlled spatio-temporal delivery device comprises a wound dressing, more preferably a bandage.

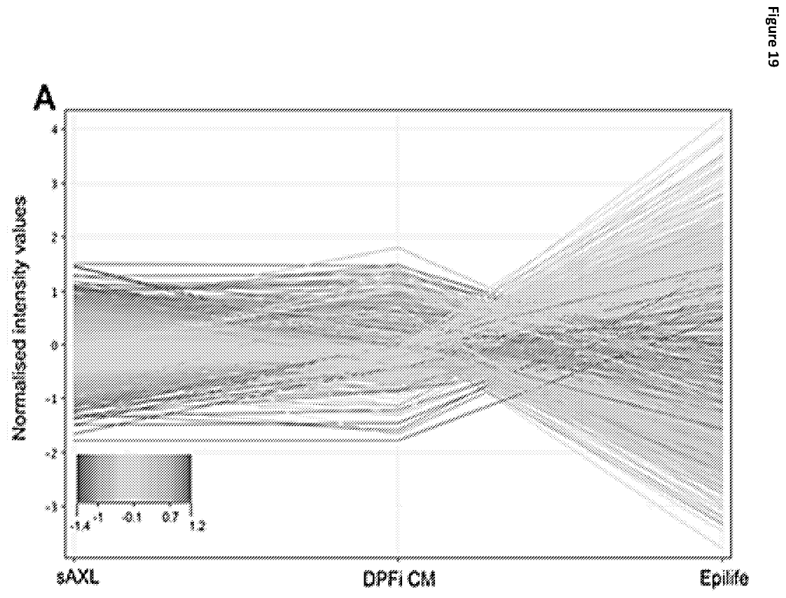

In one embodiment, the spatio-temporal delivery device is a layered bandage, an example of which is shown in FIG. 16. The layered bandage may comprise at least two layers comprising a polypeptide of the invention, wherein each layer comprises the same or different polypeptide at the same or different concentrations, wherein the polypeptide comprised in a different layer is delivered to the wound site at a different time point.

In one embodiment, the polypeptide is CCL19 in one layer and AXL in another layer; CCL19 is delivered first, preferably for up to 2 days, and AXL is delivered after CCL19, preferably for the remainder of wound closure.

The bandage layers may comprise alternate charges. The bandages may further comprise degradable material between layers enabling timed release of the polypeptides of the present invention.

Polypeptides of the invention may also be used for cosmetic purposes, due to their ability to promote cell proliferation.

Accordingly, in a seventh aspect of the invention, there is provided a cosmetic composition comprising a polypeptide selected from the group consisting of AXL, CCL19 and BMP-6, or a biologically active variant or fragment thereof.

Preferably, the cosmetic composition comprises the active domain of AXL, CCL19 or BMP-6. Preferably, the polypeptide is AXL or a variant or fragment thereof comprising the active domain of AXL.

In one embodiment, the cosmetic composition comprises AXL or a biologically active variant or fragment thereof comprising the active domain of AXL, in combination with CCL19 and/or BMP-6, or a biologically active variant thereof.

In another embodiment, the cosmetic composition comprises CCL19 or a biologically active variant or fragment thereof.

In another embodiment, the cosmetic composition comprises BMP-6 or a biologically active variant or fragment thereof.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-13 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, SEQ ID Nos:1 to 8.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1 shows fibroblast sub-types found in human scalp skin;

FIG. 2 summarises known fibroblast responses during wound healing;

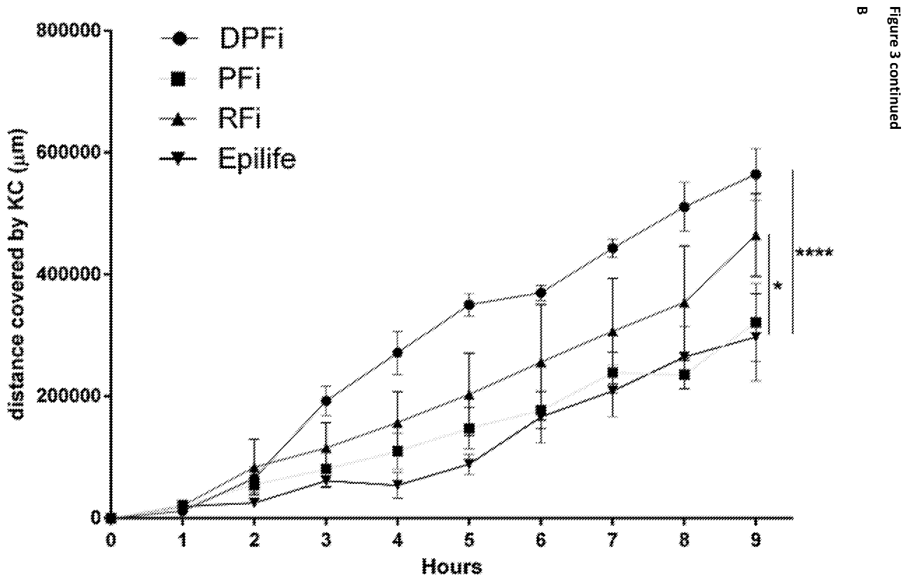

FIG. 3: A) Images of keratinocytes scratch wounds in DPFi CM, PFi CM, RFi CM and Epilife media. The red dotted line separates the cell area from the cell free area. B) DPFi promote significantly faster closure of keratinocyte scratch wounds compared to unconditioned Epilife medium. The effect is observed 4 hours after scratching, although only endpoint significance is shown on the graph for clarity. The Y-axis shows collective migration distance of all keratinocytes. N=3;

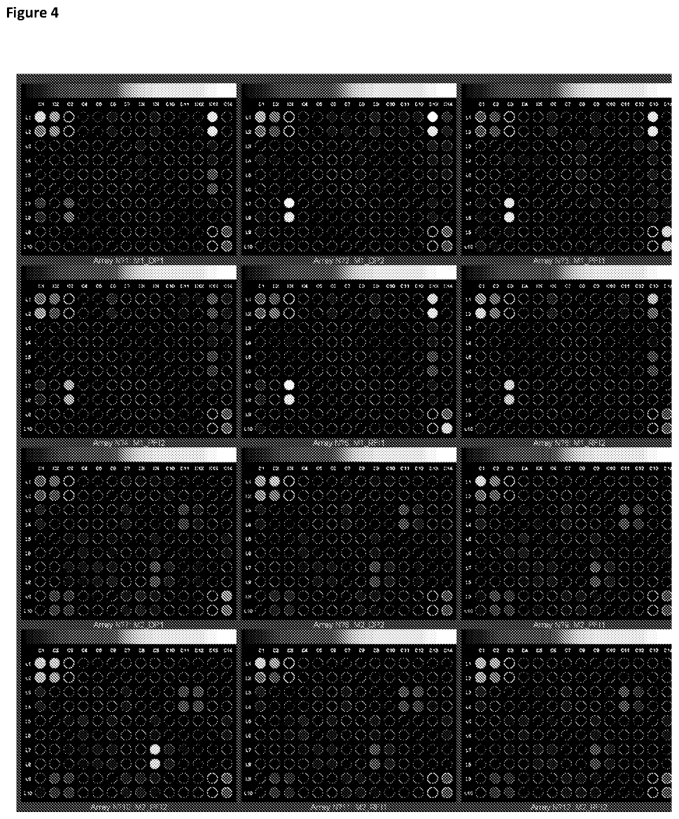

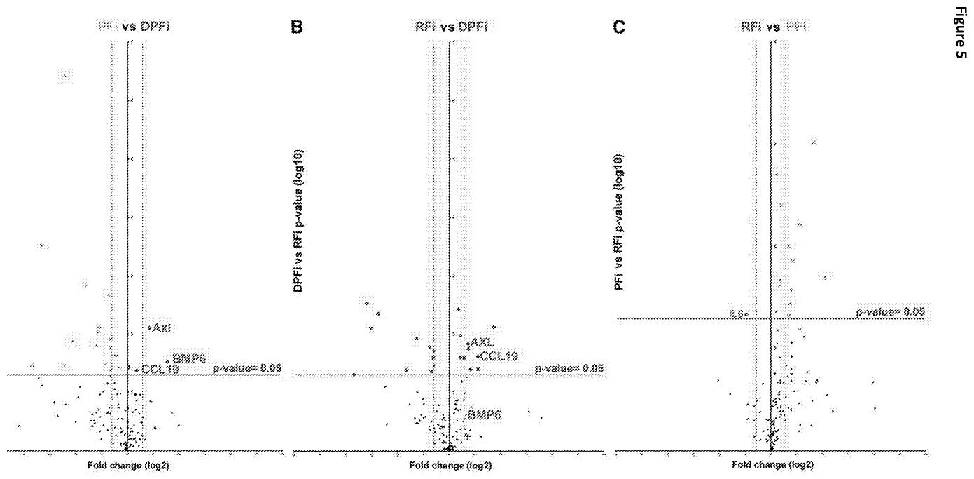

FIG. 4 shows cytokine array data—Raw Data Normalised. (A) Membrane 1 of the cytokine antibody array with DPFi CM from patient 1. (B) Membrane 1 of the cytokine antibody array with DPFi CM from patient 2. (C) Membrane 1 of the cytokine antibody array with PFi CM from patient 1. (D) Membrane 1 of the cytokine antibody array with PFi CM from patient 2. (E) Membrane 1 of the cytokine antibody array with RFi CM from patient 1. (F) Membrane 1 of the cytokine antibody array with RFi CM from patient 2. (G) Membrane 2 of the cytokine antibody array with DPFi CM from patient 1. (H) Membrane 2 of the cytokine antibody array with DPFi CM from patient 2. (I) Membrane 2 of the cytokine antibody array with PFi CM from patient 1. (J) Membrane 2 of the cytokine antibody array with PFi CM from patient 2. (K) Membrane 2 of the cytokine antibody array with RFi CM from patient 1. (L) Membrane 2 of the cytokine antibody array with RFi CM from patient 2;

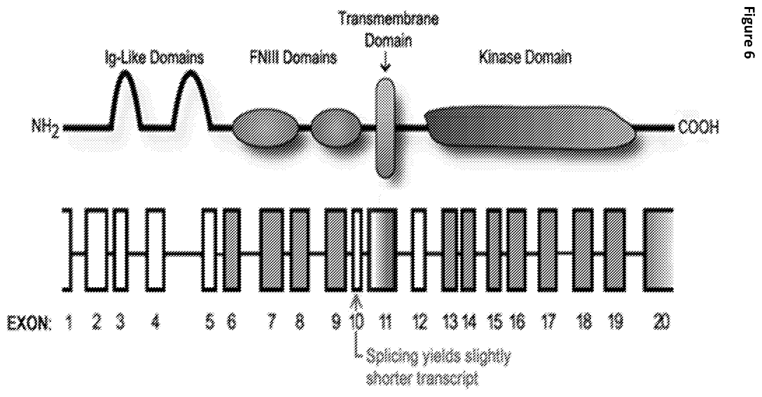

FIG. 5 shows Volcano plots of cytokine array data identifies Axl, CCL19 and BMP6 as unique to DPFi compared to PFi; (N=2)

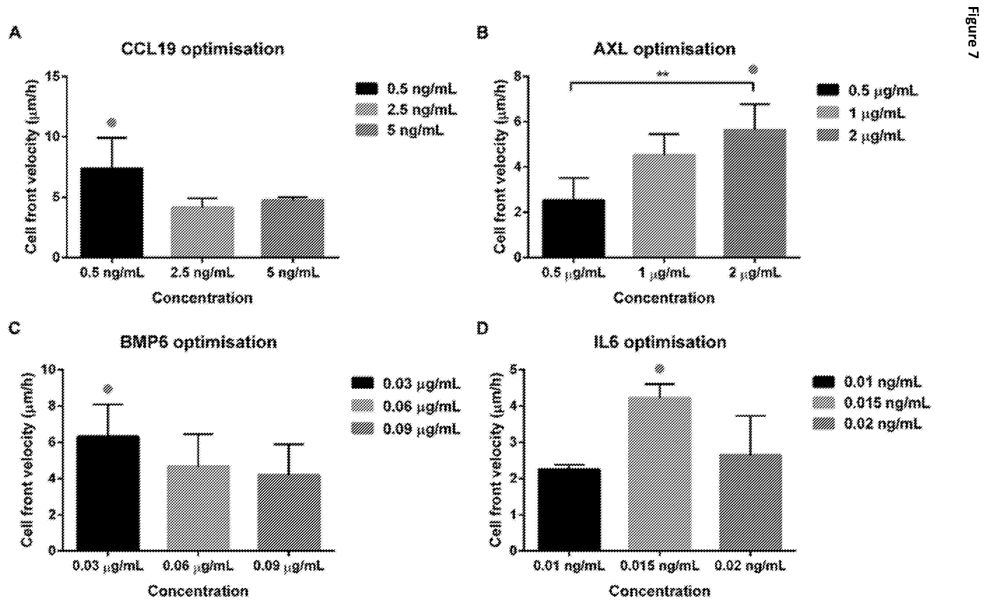

FIG. 6 shows AXL protein structure. In the inventors experiments, the extracellular domain of AXL was used, also known as soluble AXL (sAXL). Image taken from http://atlasgeneticsoncology.org/Genes/AXLID733ch19q13.html;

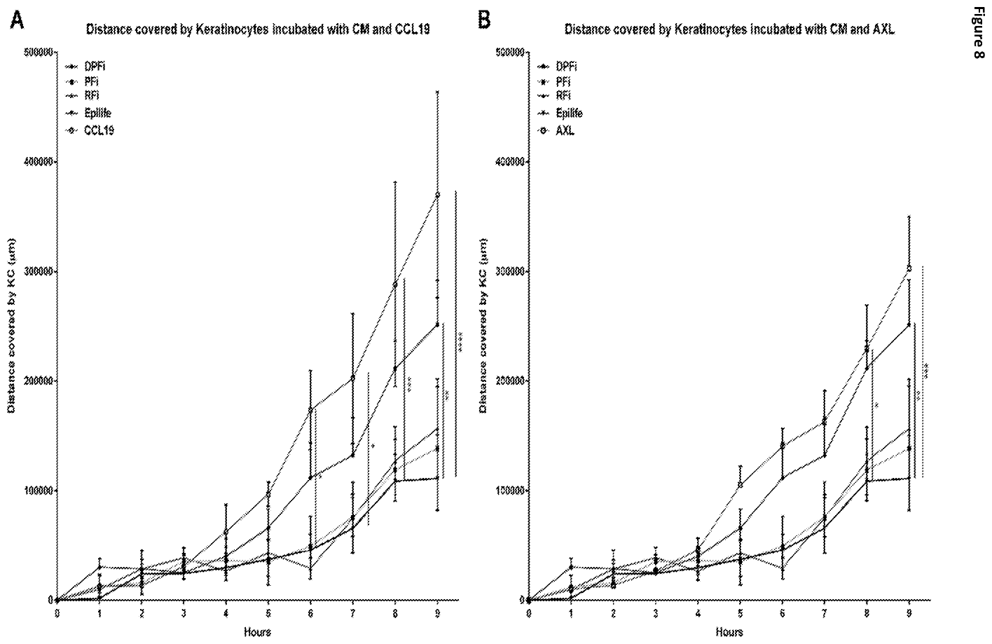

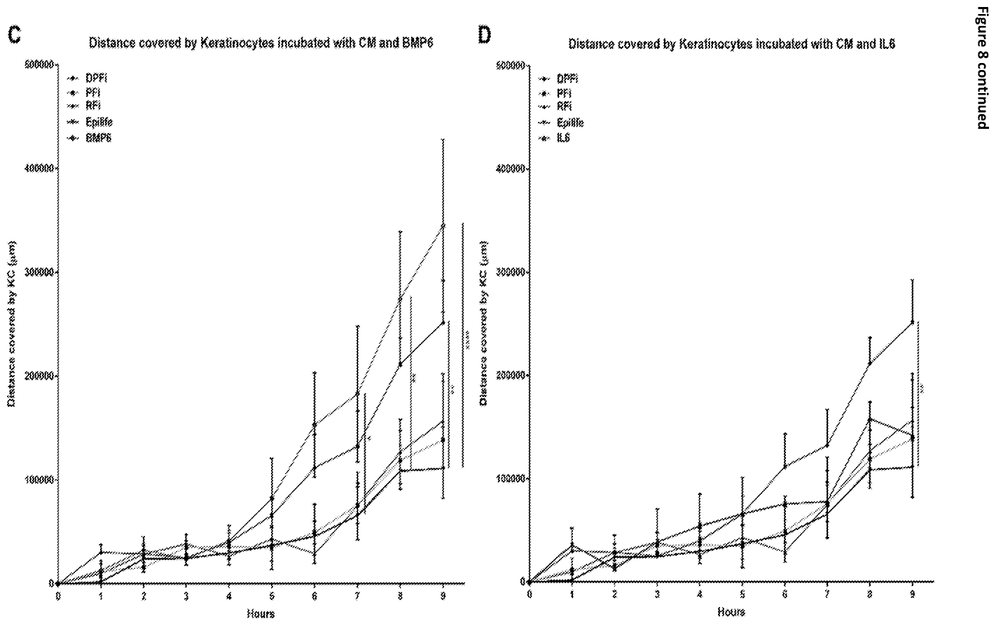

FIG. 7 shows cell front velocities for 3 cytokines concentrations for CCL19 (A), AXL (B), BMP6 (C) and IL6 (D);

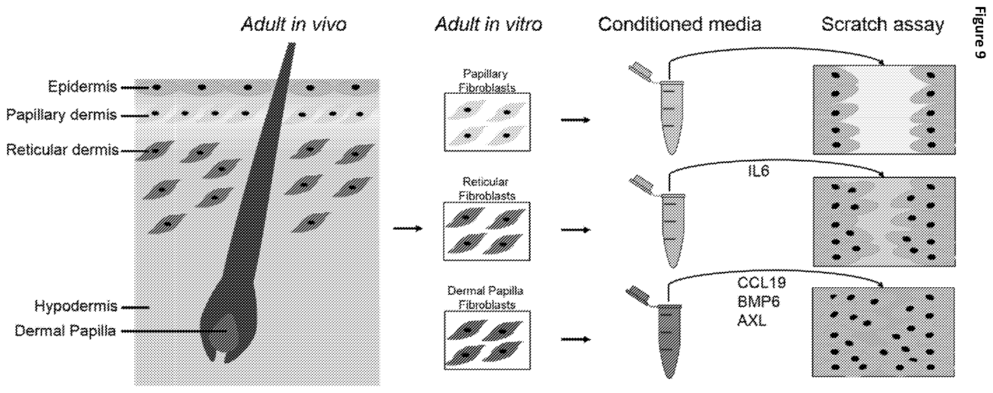

FIG. 8 shows the effect of CCL19 (A), AXL (B), BMP6 (C) and IL6 (D) on keratinocytes wound closure. The Y-axis shows collective migration distance of all keratinocytes;

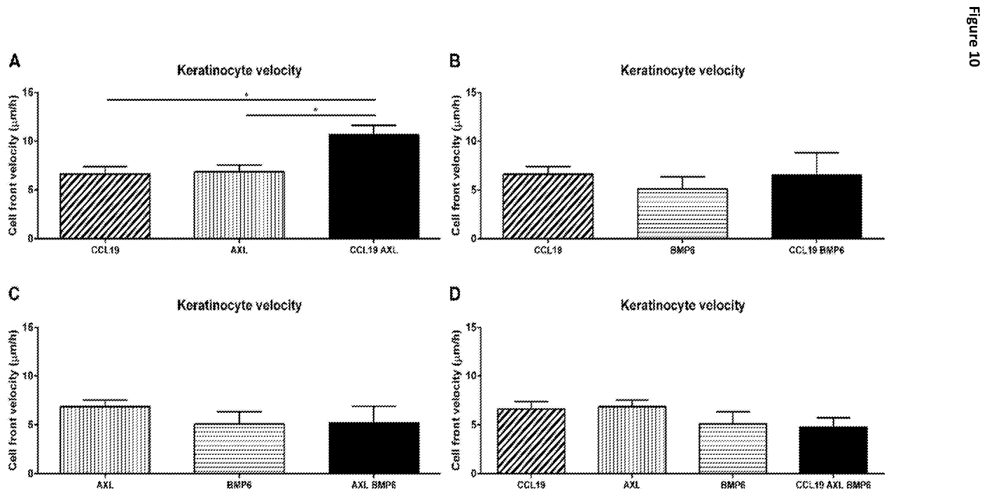

FIG. 9 shows a schematic summarising the role of fibroblasts and cytokines in wound healing;

FIG. 10 shows the evaluation of keratinocyte velocities across a wound with combinations of cytokines;

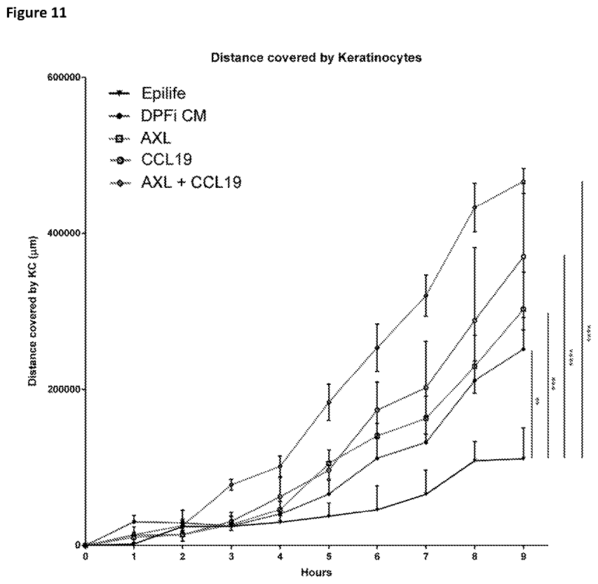

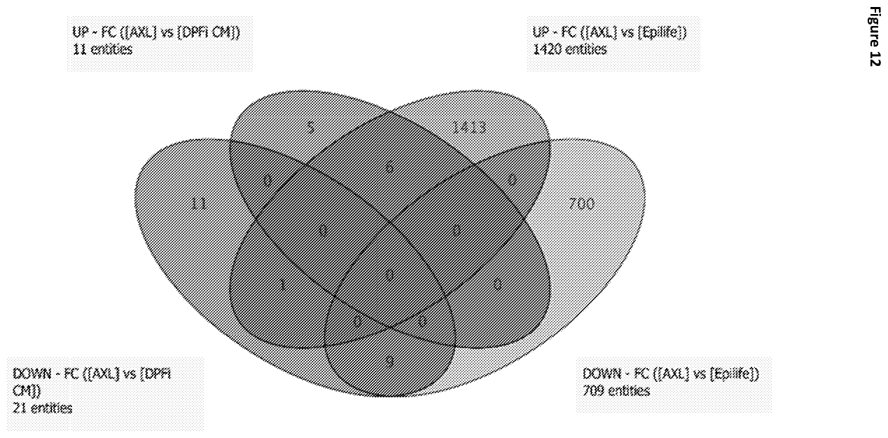

FIG. 11 shows cytokines AXL, CCL19, individually and together promote significantly faster closure of keratinocyte scratch wounds compared to unconditioned Epilife medium. The Y-axis shows collective migration distance of all keratinocytes. N=3;

FIG. 12 is a Venn diagram of significantly and differentially regulated transcripts in scratched keratinocytes exposed to AXL, DPFi conditioned medium or Epilife;

FIG. 13 shows A) the extracellular domain of AXL can bind Gas6 and B) the extracellular domain of AXL can bind itself. Taken from (Korshunov, 2012);

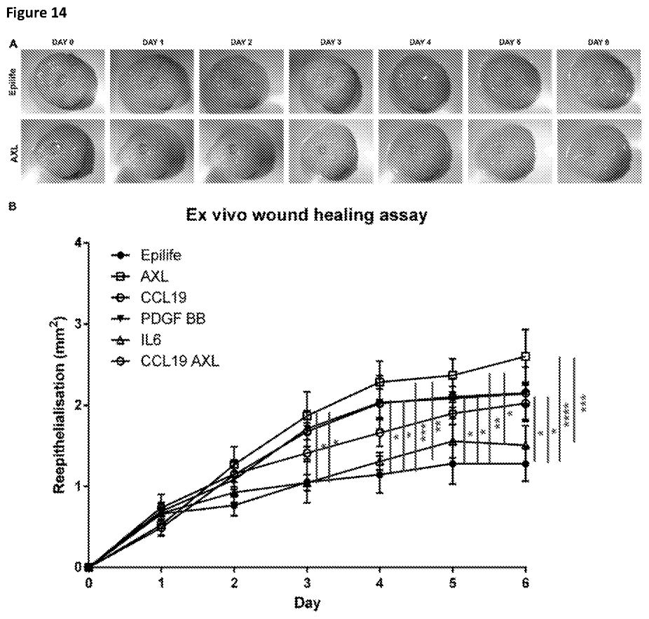

FIG. 14 shows A) examples of the punch within a punch wound closure over 6 days and B) daily delivery of AXL promotes the fastest wound closure in an ex vivo human wound. Wound closure with AXL is faster than with PDGF-BB, which is currently used to promote closure of chronic skin wounds;

FIG. 15 shows a schematic representation of the predicted AXL protein structure and showing a splice variant of the AXL polypeptide. The immunoglobulin (IgL) and fibronectin III (FNIII) domains are indicated with arrows. The amino acid sequence of AXL between the final FNIII domain and the transmembrane domain is shown to the right. The boxed 9 amino acids correspond to the differentially spliced AXL.

FIG. 16 shows an example of AXL and CCL19 temporally delivered via a layer-by-layer assembled bandage.

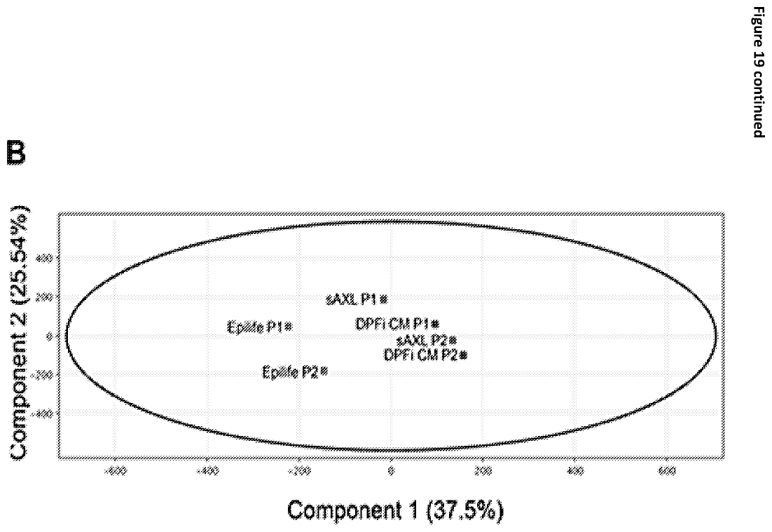

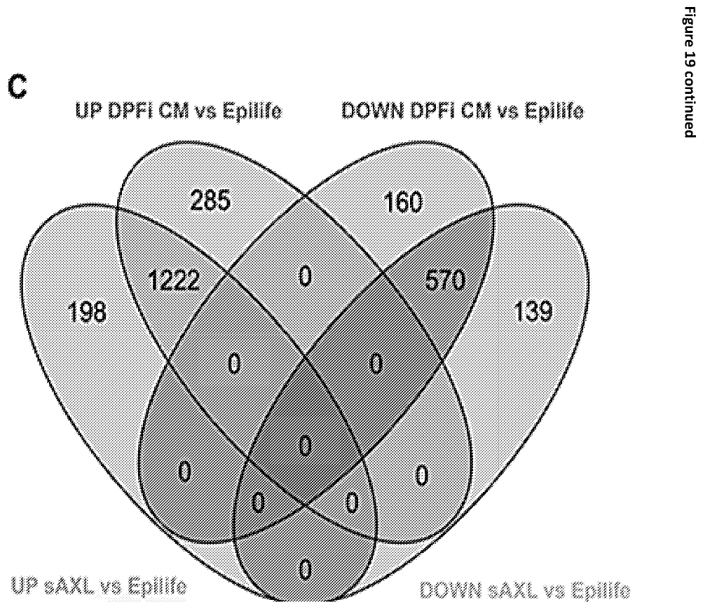

FIG. 17 shows the % of total COL1 fibres in unwounded skin, treated with the control Epilife, and treated with 2 μg/ml sAXL. Significance is displayed in the graph (P≤0.05=*, P≤0.01=**, P≤0.001).

FIG. 18 shows the results of the soft agar colony formation assay to assess sAXL carcinogenicity in vitro.

FIG. 19 shows normalised intensity values of 2574 genes differentially expressed in KC in response to sAXL, DPFi CM and Epilife. (B) PCA plot showing variance on two components. Component 1 shows treatment variance whereas component 2 shows biological sample variance. (C) Four-way Venn of the upregulated and downregulated genes in sAXL and DPFi CM versus Epilife. (D) RT-PCR analysis on an in vitro wound assay. (E) Top pathways involved in regulating wound closure. Significance is displayed in the graph (P≤0.05=*, P≤0.01=**, P≤0.001=***) as determined by a one-way Anova and the error bars represent mean±SD. In (A-C) N=2, n=3 and (D) N=2 and n=3.

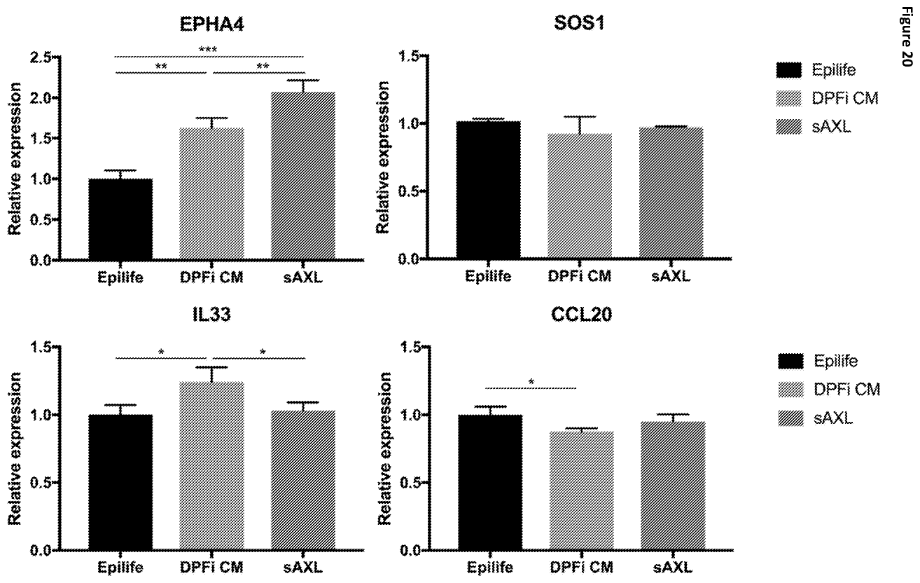

FIG. 20 shows RT-PCR data from the edge of the wound of day 3 samples of ex vivo punch assays using EPHA4, SOS1, IL33 and CCL20 primers. Significance is displayed in the graph (P≤0.05=*, P≤0.01 =**, P≤0.001=***) as determined by a one-way Anova and the error bars represent mean±SD. N=2 and n=3.

### EXAMPLES

Materials and Methods

Human Skin Biopsies

For all the in vitro experiments, cells isolated from occipital scalp skin biopsies were used. These were taken from the occipital scalp of patients undergoing surgical proceedings after receiving informed consent, and using IC-REC approved consent forms. Tissue is held under ICHTB HTA license 12275, and used in the ICHTB approved project R15055.

For ex vivo experiments, human abdominal skin with adipose tissue was purchased from Caltag Medsystems LTD.

Isolation and Cell Culture of Fibroblasts

For cell isolation scalp skin was washed in Dulbecco's minimal essential medium (dMEM; Gibco Life Technologies) with 2% Antibiotics-Antimycotics (2% ABAM; Gibco Life Technologies) for 20 minutes for cleaning prior to dissection. Using a sterile Pasteur pipette, 8 small drops, and 1 larger drop of DMEM supplemented with 1xABAM are placed onto an inverted lid of a petri dish. Each small drop is used to hold a single end-bulb for inversion in. The drops are covered by placing the base of the petri dish inside the lid. The fat and connective tissue around the lower follicle is removed using scissors. Under a stereo microscope the end bulb of the hair follicle is visible; this is carefully cut off using sterile scissors and placed into dMEM with 1% ABAM. With a fine needle (27Gx3/4″) the end of the hair follicle is fixed in place, while another needle is used to invert the end bulb structure and expose the dermal papilla containing dermal papilla fibroblasts (DPFi). The dermal papilla is then separated from the inverted end bulb and transferred into a 35 mm tissue culture dish covered in dMEM with 1% ABAM and 20% Fetal Bovine Serum (FBS; Gibco Life Technologies). The plates are placed in the incubator at 37° C., 5% CO2 and left undisturbed for 10 days during which time the papillae collapse and DFPi grow from the papilla in an explant.

With the remaining piece of skin the hypodermis is cut off to clean up the tissue. Using a scalpel blade to cut very close to the epidermis, the papillary and reticular dermis were separated into two pieces. Any remaining hair fibres in either piece of skin are removed with watchmaker's forceps. The pieces of skin are placed into separate 35 mm dishes and chopped into small pieces using scissors, and equally distributed around the dry dish. Once the tissue pieces have adhered to the base of the dish (usually 5 minutes later), dMEM containing 20% FBS and 1% ABAM is added to each dish to cover the tissue pieces and the dish is transferred to an incubator. After 10 days, cells have migrated from the reticular and papillary pieces of skin. These are termed reticular fibroblasts (RFi) and papillary fibroblasts (PFi) respectively.

Keratinocyte Isolation and Culture

Scalp skin is washed in dMEM with 2% ABAM for 20 minutes for cleaning prior to dissection. The adipose tissue is cut off the skin, and the rest of the tissue is placed in Dispase (Gibco Life Technologies) solution overnight at 4° C. After the overnight incubation, using sterile forceps, the epidermis is peeled off the dermis and was placed in 5 mL 1% Trypsin in a waterbath, at 37° C. The solution is shaken every 5 minutes to ensure that the cells are freed from the epidermis. The reaction is quenched using 5 mL Defined Trypsin Inhibitor (DTI; Gibco Life Technologies). A cell strainer with 40 μm pore sized is used to remove any pieces of tissue. The cells are then centrifuged into a pellet at 200× g for 8 minutes. The supernatant is removed and Epilife (Gibco Life Technologies) with Epilife Defined Growth Supplement (EDGS; Gibco Life Technologies) and 1% ABAM are added to the cells. The cells, which are epidermal keratinocytes (KC) are then plated at a density of 5000 cells per cm2 in flasks that have been pre-coated using a coating matrix kit (Gibco Life Technologies), which contains collagen I.

Conditioned Media Collection

DPFi, PFi and RFi cells from human occipital scalp are seeded at a density of 6000 cells per cm2 in Dulbecco's minimal essential medium (dMEM; Gibco Life Technologies) supplemented with 10% Fetal Bovine Serum (FBS; Gibco Life Technologies). After 24 hours, the cells are washed two times with Phosphate Buffered Saline (PBS; Gibco life technologies) and Epilife (Gibco Life Technologies) supplemented with Epilife defined growth supplement (EDGS; Gibco Life Technologies), which is a KC growth media, is added to the cultures. Epilife media conditioned by the DPFi, PFi or RFi is collected 2 days later. The media is then filtered through a 0.22 μm pore sized filter to remove cell debris and aliquoted and stored at −20° C. until used. Unconditioned Epilife media is subject to the same treatment and used as a control.

Keratinocyte Wound Healing Assays Using Fibroblast Conditioned Media

6 well plates are prepared by coating them using the coating matrix kit (Gibco Life Technologies) as described in KC isolation and culture section. The assay is performed by ‘wounding’ the cells using a p200 pipette.

KC are seeded at a density of 6000 cells per cm2 using Epilife supplemented with EDGS. When they reached confluency, a p200 pipette tip is used to scratch the middle of the well to create a ‘wound’ in the cells. The cells are then washed two times with PBS. Conditioned media obtained from DPFi, PFi and RFi as well as a control with just Epilife supplemented with EDGS is placed onto scratched KC. Photographs are taken at 10 timepoints, from time 0 to 9, using a phase contrast microscope at ×5 magnification. Images are analysed using Image J software.

Scratch Assay Analysis

The 9 hour measurement is used to provide information about migration and velocity. Ten images of scratch wounds closing are taken, at equal time intervals (1 hour), between the starting and end point. Optimally, the wound is not closed by the last timepoint, as migration stops when the gap reaches confluency. In order to quantify the characteristics of cell migration, images were analysed with the image processing software Image J.

The images are loaded onto the software, and the scale is set for the correct magnification of the images. Then for each timepoint, the gap is measured and calculated in μm. The difference in μm² of the area covered by the keratinocytes, is calculated by subtracting the total wounded area of each timepoint from the first to ensure consistency between results.

The cell front velocity of each wound is calculated as follows:

1. The total area the image is calculated in μm².

2. Then, the total area is multiplied by the speed of the gap closure calculated as a % per hour. 3. The total area % is divided by the length of the picture in μm to calculate how many μm per hour the front is migrating.

4. As there are two cell fronts, the μm per hour is divided by 2, to obtain the normalized cell front velocity in μm per hour.

The difference in μm of the distance covered by the keratinocytes is analysed using a two-way Anova to determine significance, and plotted as a line graph.

Human Cytokine Antibody Array

RayBio® C-Series human cytokine antibody array C1000 (RayBiotech) is used to analyse the conditioned media obtained from DPFi, PFi and RFi to determine the components of the medias. The protocol and the reagents used are ones provided by the kit supplier. All the solutions are prepared according to the manufacturer's instructions.

Antibody arrays are carefully removed from the plastic packaging and each membrane was placed (printed side up) into a well of the incubation tray provided. One membrane is used per conditioned media analysed. 2 ml of blocking buffer is pipetted into each well and incubated for 30 minutes at room temperature. The blocking buffer is then aspirated from each well. 1 ml of conditioned media is placed into each well and incubated overnight at 4° C. on a rocking plate. The next day, the conditioned media is aspirated from each well. 2 ml of 1× Wash Buffer I is added into each well and incubated for 5 minutes at room temperature. This is repeated two more times for a total of 3 washes using fresh buffer aspirating out the buffer completely each time. Then, 2 ml of 1× Wash Buffer II is added into each well and incubated for 5 minutes at room temperature. This is repeated one more time for a total of 2 washes using fresh buffer and aspirating out the buffer completely each time. 1 ml of the pre-prepared Biotinylated Antibody Cocktail is into each well and incubated overnight at 4° C. The next day, 2 ml of 1× HRP-Streptavidin is added into each well and incubated overnight at 4° C. HRP-Streptavidin is aspirated from each well. The membranes are then washed with 2 ml of 1× Wash Buffer I and incubated for 5 minutes at room temperature. This is repeated two more times for a total of 3 washes using fresh buffer and aspirating out the buffer completely each time. Then, 2 ml of 1× Wash Buffer II is added into each well and incubated for 5 minutes at room temperature. This is repeated one more time for a total of 2 washes using fresh buffer aspirating out the buffer completely each time. The membranes are transferred, printed side up, onto a sheet of tissue paper lying on a flat surface. Excess wash buffer is removed by blotting the membrane edges with another piece of paper. The membranes are transferred, printed side up, onto a plastic sheet provided, lying on a flat surface. Into a single clean tube, equal volumes (1:1) of Detection Buffer C and Detection Buffer D are added and mixed well with a pipette. The Detection Buffer mixture is then gently pipetted onto each membrane and incubated for 2 minutes at room temperature. Exposure should ideally start within 5 minutes after finishing the last step and completed within 10-15 minutes as chemiluminescence signals will fade over time. Another plastic sheet is placed on top of the membranes by starting at one end and gently rolling the flexible plastic sheet across the surface to the opposite end to smooth out any air bubbles. The membranes are ‘sandwiched’ between the two plastic sheets. The sandwiched membranes are transferred to the chemiluminescence imaging system to expose for 1 minute.

Human Cytokine Array Analysis

The protein analyser plugin for Image J is used to analyse the cytokine array antibody membranes. Images can be loaded individually onto the software and the analysis is performed using the “Array Analysis Menu” followed by the “Array Analysis” function. This action proposes a method of background subtraction and builds a graphical interface for the dot matrix analysis. The visualisation can then then optimised by activating some options available from the graphical interface. Once the mask is set and recognises the membrane, a grid will form, and the matric can be measured automatically as a table of values.

Once the arrays are individually analysed using the “Array Analysis” tools, the “Group Pattern” menu then allows the user to obtain a global view of a set of arrays. The parent folder is set to contain the analysed arrays. This folder is selected containing the array analyses by the “Masterize from Analysis Repertories” function. This function looks for result tables coming from the “Array Analysis” functions, in the parent folder. The tool explores any sub-levels, and builds a master image, or pattern, associated to a master table presenting all the results. The program exhibits two default master representations:

1. The default master pattern presents the arrays as they came from the analysis, with the visualization scaled between zero and the maximum values encountered in each array.

2. The initial normalized pattern presents a normalization between zero and the maximum value found in the master. This representation gives the most natural aspect of the modelled pattern compared to the initial images.

The masters were then normalised using the internal references provided by the manufacturer on the membrane as positive and negative controls, by using the “Group Pattern Menu” and “Set Internal Control and References”. Each value is normalized following this formula:

Dot Value norm=(Dot Value−mean(Controls))/mean(References).

Once the normalised values are obtained, the following three correlations can be made: DPFi vs PFi, DPFi vs RFi and PFi vs RFi by calculating the fold change (log2) and the p-value (log10) by using t-test with unequal variance. The cut off values of p-value<0.05 and fold change ranging from −0.5 to 0.5 are set

Optimisation of Cytokine Concentrations and Cytokine Scratch Assay

The following four recombinant human cytokines were chosen to assess their effect on keratinocyte migration; AXL receptor tyrosine kinase (AXL; R&D systems), Chemokine ligand 19 (CCL19; Biolegend), Bone morphogenic protein 6 (BMP6; Biolegend) and Interleukin 6 (IL6; Gibco, life technologies). The specific activity of each cytokine is specified as a range by the supplier and three concentrations at the top, bottom and middle of this range were tested to obtain the concentration that yields the fastest wound gap closure using the KC scratch assay as previously described. The optimal cytokine concentrations were then used individually or in combination with one another, in the in vitro scratch wound assay. The following combinations were assessed: AXL, BMP6, CCL19, AXL+BMP6, AXL+CCL19, BMP6+CCL19, AXL+CCL19+BMP6, and IL6.

Recombinant human PDGF-BB (Biolegend) was also purchased to assess its effect on KC reepithelialisation. This cytokine has been optimised in human fibroblasts in culture before and its maximum effect was recorded to be 5 ng/ml, therefore this concentration was used going forward.

Human Ex Vivo Wound Model

Human abdominal skin with adipose tissue is obtained with informed consent. Subcutaneous fat is removed to obtain a sheet of epidermis with a thin dermis below. A series of 2 mm diameter partial thickness wounds are made using a biopsy punch, and the epidermis and papillary dermis are removed from these punches using fine scissors. Surrounding these 2 mm punches, a series of 8 mm full wounds are made, to create a series of wounds within a punch to assess wound closure of the 2 mm wound within the 8 mm punch. These 8 mm punches are then transferred to the top of a non-woven gauze and a 0.45 μm nylon membrane (Millipore) in a 6 well plate. 1.5 ml of William's E media (Life Technologies) supplemented with 1% P/S, 2 mM L-Glutamine (Gibco), 10 μg/ml Insulin (Sigma) and 10 ng/ml Hydrocortisone (Sigma) is added onto the non-woven gauze in each well. In order to test the effect of the different cytokines in wound closure, 6 conditions are tested simultaneously, with at least 6 technical replicates for each condition. The conditions tested are AXL, CCL19, PDGF-BB, IL6, CCL19+AXL and Epilife supplemented with EDGS. 5 μl of solutions of these cytokines in Epilife with EDGS (control) are pipetted daily into the centre of the wound. Media is also changed daily with excess media being removed from the well and replaced with 1 ml fresh media. In the experiment as shown in FIG. 17, conditions were tested simultaneously (sAXL and Epilife), with 6 technical replicates for each condition. 5 μl of solutions of 2 ug/ml of sAXL (R&D systems) in Epilife complete media and the control Epilife complete media were pipetted daily into the centre of the wound. Media was changed daily with excess media being removed from the well and replaced with 1 ml fresh media. 6 days after wounding, the ex vivo skin was embedded in OCT and was sectioned at 80 μm thick sections to be imaged via SHG.

Images are taken of the wounds every 24 hours with a stereo microscope until wound closure is achieved (usually 5-10 days). The images are analysed using Image J. A two-way ANOVA can be used to analyse the difference in mm between pictures (indicating closure) and comparisons between conditions are made at individual time points using the same test in Graphpad Prism 6.0.

Transcriptional Analysis to Determine the Effect of AXL and DPFi CM on Keratinocyte Gene Expression

3 wells of a 6 well plate were prepared by coating them using the coating matrix kit (Gibco Life Technologies) as previously described. KC are seeded at a density of 6000 cells per cm2 using Epilife supplemented with EDGS. When they reached confluency, a p200 pipette tip is used to scratch the well in 4 different regions (in a hashtag pattern), to create a ‘wound’ in the cells. The cells are then washed two times with PBS. Conditioned media obtained from DPFi, AXL, and control with just Epilife supplemented with EDGS are placed on wounded cells. After 6 hours, media is removed, cells are washed in PBS, then RNA is collected using the RNeasy Plus Micro Kit (Qiagen). RNA is used to synthesized first-strand complementary DNA (cDNA) which is then converted to double-stranded cDNA, and used as a template for in vitro transcription generating cRNA. The cRNA is then transferred for hybridization and scanning onto the GeneChip™ Human Genome U133 Plus 2.0 Array.

Microarray Computational Analysis

Raw data from the microarray are analysed using the commercial software package Genespring GX 14.9 (Agilent Technologies Inc.). The intensity values of the samples are normalised and summarised using RMA algorithm. Parametric tests, with the p-value set at 0.05 are performed to determine significant differential expression between samples. Entities are chosen on a fold change cut off of >=2. Venn diagrams enable identification of genes which are uniquely upregulated or down regulated in keratinocytes after exposure to AXL, but not Epilife or DPFi conditioned medium. Pathway analysis on these specific genes is performed in Ingenuity.

Second Harmonic Generation (SHG)

To compare changes in the total collagen type 1 fibres, 80 μm sections of ex vivo skin were imaged. 30 μm-deep tile scans (6× Z-stack steps) of approximately 3,000 μm×2,000 μm were obtained by the Second Harmonic Generation (SHG) by imaging the healed dermis. All images were first processed by adjusting the min-max at (0, 3000). Four regions of interest (ROIs) of 600 μm×450 μm were then selected from tile scans within the previously wounded area or control area (unwounded). Stacks were then separated into single images. An interactive learning and segmentation toolkit called ilastik was applied to facilitate further analysis. As part of the pixel classification workflow, two features: fibres, and background (no signal), were selected and used for training of a set of 6 images. The probability mask was then applied to 20 images (single Z-stack steps) of each time point in a batch processing mode. The inventors then used the FIJI Macro Recorder to automatize the separation of segmented channels and quantification of the pixel area. Based on the pixel area covered by the two features, the total proportion of collagen in the dermis was calculated.

Soft Agar Colony Formation Assay

The method used for this assay has been previously described in detail by Borowicz et al 2014. Briefly, using a 6 well plate, a bottom layer of agar was plated by adding 1:1 ratio of 2× DMEM 10% FBS and 1% noble agar solution. The plates were covered, and the agar mixture was left to solidify at room temperature in the cell culture hood for 30 minutes. Once the lower layer of agar has solidified, the upper agar layer was prepared. The cells were seeded at 10000 cells/well and were resuspended in 1× DMEM 10%FBS and 0.6% agar in a 1:1 ratio and added on top of the solidified lower agar layer. The upper layer was left to solidify at room temperature in the cell culture hood for 30 min before placing into a 37 ° C. humidified cell culture incubator. A layer of medium was maintained over the upper layer of agar which contained the different concentrations of sAXL or the control media. 100 μl of medium was added twice weekly for 21 days. After 21 days the cells were stained by adding 200 μl of nitroblue tetrazolium chloride solution per well and incubating plates overnight at 37 ° C. The plates were then imaged to visualise colony formation.

Transcriptional Analysis

6 well plates were prepared by coating them using the coating matrix kit. KC from two patients were seeded at a density of 6000 cells/cm2 using Epilife supplemented with EDGS. At confluency, a p200 pipette tip was used to scratch the well in 4 different regions (in a hashtag), to create a ‘wound’ in the cells. The cells were then washed two times with PBS to remove debris. CM obtained from DPFi, sAXL, and control with just Epilife supplemented with EDGS were added on the wounded cells. After 6 hours, media was removed, cells were washed in PBS, then RNA was collected using the RNeasy Plus Micro Kit (Qiagen). RNA was used to synthesize first-strand complementary DNA (cDNA) using Nugen Ovation V2. This was then converted to double-stranded cDNA, and used as a template for in vitro transcription to generate cRNA using the Nugen Encore Biotin Module. The cRNA was then transferred for hybridization and scanning onto the GeneChip™ Human Genome U133 Plus 2.0 Array. Raw data from the microarray was analysed using the commercial software package Genespring GX 14.9 (Agilent Technologies Inc.). The intensity values of the samples were normalised and summarised using RMA algorithm. Parametric tests, with the p-value set at 0.05 were performed to determine significant differential expression between samples. Entities were chosen on a fold change cut off of >=2. Venn diagrams enabled identification of genes which were uniquely upregulated or down regulated in KC after exposure to sAXL and DPFi CM, but not Epilife. Pathway analysis on these specific genes was performed using Ingenuity Pathway Analysis (IPA; Agilent).

mRNA Extraction, Reverse Transcription and RT-PCR

RNA extraction was performed using a QiaShredder and RNeasy Mini kit (Qiagen) following manufacturer's instructions to obtain RNA from fresh tissue, DPFi, PFi and RFi. cDNA was synthesised using OligoDT primers and SuperScript III (Life Technologies). For the RT-PCR, PowerUP SYBR Green Master Mix (2X; Life Technologies) was used with primers designed using the UCSC database. RT-PCRs were run on an ABI 7500 Fast RealTime PCR with the cycles as follows: 2 minutes at 50° C. and 2 minutes at 95° C. followed by 35 cycles of 15 seconds at 95° C. and 1 minute at 60° C. Expression analysis was performed relative to GAPDH using the ddCT algorithm, with expression in fresh tissue used as a baseline comparison (value=1). RT-PCR was performed using cDNA from two biological replicates, and the relative expressions were consistent in both patients. Statistical analysis was performed using one-way Anova test.

Statistical Analyses

The number of replicates used for each experiment is indicated in their respective figure legends where N is the number of biological replicates and n is the number of technical replicates. Data are presented as the mean and standard deviation. Statistical significance was assessed using one-way ANOVA and a Tukey multiple-comparison post-hoc test unless otherwise stated. Differences were considered statistically significant if their p value≤0.05.

Results

### Example 1

### Hair Follicle Derived Fibroblasts Accelerate Wound Closure In Vitro

First, to assess whether sub-types of fibroblasts have differential paracrine effects on epidermal keratinocytes, the inventors collected keratinocyte medium (Epilife) which was conditioned by 3 sub-types of fibroblasts (DPFi, PFi and RFi) for 48 hours, filtered it to remove cell debris, and placed onto keratinocytes. Keratinocytes were scratched with a pipette, and the migration of cells into the scratch wound was then assessed. In this well established in vitro wound healing assay (13), the inventors found that RFi conditioned medium promoted significantly faster (p<0.05) wound closure compared to unconditioned keratinocyte medium. This supports the inventors' working hypothesis, that RFi release growth factors can promote re-epithelialisation. However, the most surprising result came with the DPFi conditioned medium, which promoted significantly faster (p<0.001) migration of keratinocytes across the scratch wound (FIG. 3) compared to controls and other fibroblast conditioned medias.

### Example 2

### Identification of Components of DPFi Medium Which Promote Wound Healing

To identify which factors are released by cells and contributing to the observed effects, the inventors then conducted cytokine arrays (FIG. 4) on keratinocyte medium conditioned by DPFi, RFi and PFi. The inventors then performed differential analysis to identify cytokines which were significantly released by DPFi compared to RFi or PFi (FIG. 5). The inventors identified 3 factors (AXL, CCL19, BMP6), which were released into the culture medium by DPFi at significantly higher levels than PFi. They also identified 10 factors released into the medium by DPFi at significantly higher levels than RFi, including AXL and CCL19 which were previously identified in the DPFi vs PFi cytokine array. BMP6 was released from DPFi at higher levels than from RFi, but did not pass the significance threshold. IL6, a well-known regulator of wound healing and epithelial migration (14, 15) was found at significantly higher levels in the RFi conditioned medium compared to the PFi, and the inventors, although not wishing to be bound by hypothesis, postulate that this may be contributing to the observed accelerated closure with RFi conditioned medium.

### Example 3

### Use of AXL in the Laboratory

For CCL19, BMP6 and IL6, it was easy to purchase a peptide. However, surprisingly, further research into AXL revealed that it is actually a tyrosine kinase receptor protein, and it was initially confusing as to why a transmembrane protein was on the cytokine array. The inventors have found that the extracellular domain of AXL is cleaved by ADAM10, leaving a small peptide product. The full structure of AXL is 894 amino acids long (FIG. 7A); it is a 140 kDa glycoprotein in the TAM receptor tyrosine kinase family with the gene located on chromosome 19q13.2 encoding 20 exons. The AXL gene is also known as UFO, ARK, JTK11 or TYRO7. Exons 1-10 encode the extracellular domain, which includes a signal peptide (aa 1-37), two immunoglobulin (Ig) domains (aa 37-124 for domain 1, 141-212 for domain 2), and two fibronectin type III (FNIII) domains (aa 224-322 for domain 1, 325-428 for domain) and is approximately 60-80 kDa (FIG. 6). Thus, as it was the extracellular domain of AXL which was detected on the cytokine array, the inventors purchased a peptide aa 33-440 of AXL for use in further experiments (FIG. 7). Exon 11 of AXL also encodes an extracellular region (aa438-451) that is subject to proteolytic cleavage along with exons 1-10 meaning the whole extracellular region of AXL is from aa1-451. Exons 12-20 compose the intracellular domain, which includes the tyrosine kinase domain (exons 13-20) (16). Not wanting to be bound to any particular hypothesis, the results described herein, and effect elicited by the soluble form of AXL, may be as a result of binding through one or both of the Ig domains, one of both of the FNIII domains, or either of the above combinations together (FIG. 7B).

### Example 4

### AXL Promotes Wound Closure in Scratch Assays

To further evaluate how the DPFi specific cytokines and IL6 might have a role in wound healing, the inventors assessed their effect on keratinocyte migration in a scratch wound individually and in combinations, compared to DPFi conditioned medium. The inventors used three concentrations, at the top, bottom and middle of the range suggested by the manufacturer, and determined maximal cell front velocity across a scratch wound for all three concentrations. The inventors identified an optimal concentration for use in further experiments (FIG. 8).

Surprisingly, when the inventors used the determined concentrations of cytokines in scratch wound assays, and assessed closure, they found that CCL19, AXL and BMP6 could all promote faster wound closure than DPFi, and significantly faster wound closure than Epilife control. IL6 on the other hand showed relatively little difference from the RFi conditioned medium in its ability to promote wound closure. This section is summarised in FIG. 9.

The inventors further assessed combinations of the cytokines together and, even more surprisingly, found that AXL by itself, CCL19 by itself, or AXL in combination with CCL19 were the best when they evaluated the maximum cell velocity front of keratinocytes crossing a scratch wound (FIG. 10). The inventors therefore used these individually, and in combination in the full scratch wound assay, plotting closure day by day and found, surprisingly, that CCL19 in combination with AXL significantly accelerated wound closure more than AXL or CCL19 by themselves (FIG. 11). Not wishing to be bound to any hypothesis, this effect could be promoted further by assessing temporal delivery of the cytokines. For example, CCL19 for 2 days, followed by AXL for the remainder of the wound closure. Not wanting to be bound to any particular hypothesis, each of these cytokines will activate distinct pathways which are important for wound closure. However, all the cytokines together at the same time may overload the cells.

### Example 5

### Role of Specific Cytokines in Wound Healing

After extensive research by the inventors, to their knowledge there hasn't been a connection previously made between AXL, the AXL cleaved peptide, nor AXL in combination with CCL19 in cutaneous wound healing or scar reduction.

As little is known about the role of the cleaved peptide of AXL, and the signalling pathways it is involved in, the inventors performed transcriptional analysis to further their understanding of it. The inventors took human keratinocytes in culture, and scratched them to create a ‘scratch wound’. Next, the inventors placed AXL (which is a component of DPFi conditioned medium), or DPFi conditioned medium, or control medium (Epilife) on cells for 6 hours before collecting RNA for analysis. After performing microarrays to identify the transcriptional profiles of cells, and identifying genes which were differentially expressed between conditions, the inventors identified 6 transcripts uniquely up regulated in AXL conditioned cells, and 9 transcripts which were down regulated (FIG. 12) (Table 1).

TABLE 1
15 transcripts identified as up or down regulated
uniquely in keratinocytes containing AXL in media.

| Affymetrix | | FC AXL vs | FC AXL vs |
|---|---|---|---|
| Probe set ID | Gene Symbol | DPFi CM | Epilife |
| 204105_s_at | NRCAM | 2.78 | 4.55 |
| 209794_at | SRGAP3 | 2.10 | 8.17 |
| 227497_at | SOX6 | 2.30 | 3.03 |
| 227498_at | SOX6 | 2.15 | 3.98 |
| 227943_at | — | 2.00 | 2.51 |
| 230343_at | CST3 | 2.01 | 8.71 |
| 205122_at | MSANTD3- | −2.17 | −4.32 |
| | TMEFF1///TMEFF1 | | |
| 205220_at | HCAR3 | −2.12 | −3.17 |
| 210517_s_at | AKAP12 | −2.80 | −6.07 |
| 211924_s_at | PLAUR | −2.32 | −9.57 |
| 216243_s_at | IL1RN | −2.03 | −4.30 |
| 227529_s_at | AKAP12 | −3.05 | −2.37 |
| 1554086_at | TUBGCP3 | −2.66 | −3.60 |
| 1555673_at | KRTAP2-3///KRTAP2-4 | −2.22 | −2.48 |
| 1566764_at | MACC1 | −2.35 | −6.61 |

The vitamin k dependent protein Gas6 is known to bind AXL and trigger autophosphorylation of the AXL cytoplasmic domain, which leads to further downstream processes such as migration, proliferation and reduced inflammation (21). It has also been suggested that AXL is able to undergo homophilic binding of its extracellular domains with AXL on neighbouring cells (FIG. 13). This is a ligand-independent type of receptor activation that occurs after overexpression of AXL (22, 23). Potentially, but not wanting to be bound to any particular hypothesis, addition of sAXL to the media is either neutralising GAS6 thereby inhibiting the AXL downstream processes or alternatively sAXL is acting as an AXL decoy and undergoes homophilic binding with membrane bound AXL on cells. Not wishing to be bound by any particular hypothesis, sAXL may either be inhibiting or activating full length AXL.

### Example 6

### Assessment of Wound Healing in a Human Skin Model

So far, the inventors had only assessed the role of AXL and CCL19 in 2D scratch wounds, and so they sought to evaluate their effect in an ex vivo human skin model, called a punch within a punch (FIG. 14A). Here, a small wound is created within a larger wound, and wound closure of the small wound is evaluated over the course of a few days (24). Epithelial migration over the wound bed can then be plotted as a function of time. Using this assay, the inventors evaluated the effect of AXL alone, CCL19 alone and in combination with AXL. They also evaluated IL6 as it has a documented role in wound healing along with PDGF-BB which is the active component in the aforementioned Regranex product. Surprisingly, the inventors found that AXL alone promoted the fastest wound closure in the ex vivo human skin wound, significantly faster than the control just 3 days after the start of the experiment (FIG. 14B). It is worth noting that AXL promoted faster wound closure than Regranex, which is currently still used in the USA to promote wound closure and thus the AXL represents a significant improvement over currently known wound treatments.

It is also important to note here that the punch within a punch assay wound was given fresh doses of cytokines daily. Not wanting to be bound to any particular hypothesis, when assessing modes of delivery, bandages assembled using a layer by layer technique, or specific cosmeceutical formulations would enable sustained delivery of cytokines over the course of a few days, which would be beneficial as a treatment option for chronic wounds. Lower concentrations of cytokines could be employed if delivery was sustained over longer time periods.

### Example 7

### Reduced Scar Formation with the Addition of sAXL Ex Vivo

The skin dermis is mainly composed of cells (such as fibroblasts and endothelial cells) and extracellular matrix (ECM). Interstitial collagens make up the majority of that ECM with Collagen I (COL1) being one of the main ECM protein in the skin dermis (Xue and Jackson 2015). After a cutaneous injury, the skin heals via a series of events known as haemostasis and inflammation, reepithelialisation and ECM remodelling. Dermal remodelling can take months to years to be completed. Previous research has shown that the content of COL1 is significantly altered in a scar tissue compared to unwounded controls, with significantly higher COL1 content in wounded patients even at 24 months post injury (Wang Cheng and Guo-an 2011). In order to quantify scar formation (COL1 content) in ex vivo skin, the inventors used Second Harmonic Generation (SHG) imaging (see methods section) to quantify percentage (%) of total COL1 fibres within the skin dermis of skin healed with and without our cytokine of interest (see methods section; FIG. 17). Analysis of the SHG images using image segmentation revealed that the volume of collagen is significantly greater in Epilife treated wounds (94% +/−SD) in comparison to the both the unwounded area (85% +/−SD) and sAXL treated wounds (89% +/−SD). Since scar tissue contains higher amounts of total COL1, the significantly lower amounts of COL1 found in sAXL, and the similarity to unwounded skin, suggest that sAXL promotes a reduced scar phenotype in human skin.

### Example 8

### Soft Agar Colony Formation Assay to Assess sAXL Carcinogenicity In Vitro

Transformation of normal cells into neoplastic cells occurs via a series of genetic alterations, leading to a cell population that is capable of proliferation in a three-dimensional environment. Anchorage-independent growth is the ability of neoplastic cells to grow independently of a solid surface. The soft agar colony formation assay (Method previously described by (Borowicz, Van Scoyk et al. 2014)) has been widely used to monitor cell transformation and anchorage-independent growth, by visualising colony formation after 3 weeks in culture. The inventors used this assay to identify whether different concentrations of sAXL could transform skin fibroblast cells from the dermis, into neoplastic ones. The inventor's results show that sAXL does not transform the cells into neoplastic cells at concentrations 2 μg/ml to 32 μg/ml, as the cells are not able to proliferate and form colonies in the three-dimensional environment. This was compared to a positive control of Suite-007 (human cancel cell line derived from the metastatic liver from Pancreatic ductal adenocarcinoma) cells which were able to form colonies in the soft agar assay in contrast to sAXL and the negative control that did not form any colonies. Here, the inventors have illustrated that the soft agar colony formation assay using sAXL at concentrations 2 μg/ml to 32 μg/ml does not promote transformation of normal cells into neoplastic cells (FIG. 18).

### Example 9

### Microarray Reveals that sAXL Promotes Keratinocyte Migration While Inhibiting Keratinocyte Differentiation

The inventors used a microarray to perform an unbiased transcriptional analysis where they compared sAXL, Dermal papilla fibroblast conditioned media (DPFi CM) and Epilife on scratch wound transcription in keratinocytes (KC) in vitro (FIG. 19A). Raw data from the microarray was analysed with a one-way Anova test identifying 2574 genes which were significantly and differentially regulated between conditions (FIG. 19A). Principal component analysis shows that sAXL and DPFi clustered more closely together than Epilife thus sharing less variance (FIG. 19B). Specifically, variance between Epilife media and both DPFi CM and sAXL was on the 1st principle component while variance between the biological repeats (P1 and P2) was on the 2nd principle component.

To help the inventors determine unique genes involved in accelerated wound closure in vitro, upregulated and downregulated gene lists of sAXL and DPFi CM vs Epilife were plotted in a Venn diagram (FIG. 19C). Using the Venn the inventors identified 1222 genes upregulated and 570 downregulated in both DPFi CM and sAXL treated KC in comparison to KC treated with the Epilife control. The inventors believe that these gene lists encompass genes which are enabling accelerated scratch wound closure as a result of their differential regulation (Table 1). In an attempt to identify the pathways activated in response to the genes uniquely upregulated/downregulated by DPFi CM and sAXL, the inventors used IPA software to identify signalling pathways activated or inhibited in the KC. Using the list of genes upregulated/downregulated in KC in both sAXL and DPFi CM, the inventors identified three main pathways that are activated; the Hippo pathway, Ephrin pathway and Epidermal Growth Factor (EGF) pathway (FIG. 19D). Activation of Yes-associated protein 1 (YAP1), a member of the Hippo pathway, can promote migration of cells while blocking KC differentiation. In addition, the EGF receptor (EGFR) was also upregulated in KC, predicted to promote cell cycle progression but simultaneously block KC differentiation. Ephrin A4 (EPHA4), a member of the Ephrin pathway, was the most highly upregulated gene in the KC in sAXL and is known to promote cell migration, cell movement and adhesion of epithelial cells.

To validate the transcriptional data, the inventors performed RT-PCR on EPHA4, SOS1, IL-33 and CCL20 (FIG. 19E and Table 1).

TABLE 1
Microarray top upregulated and downregulated genes.
Gene list with the FC (Fold Change) of the top ten upregulated and
downregulated genes in DPFi CM and sAXL compared to Epilife.
Highlighted genes have been validated both in vitro and ex vivo.

| Gene | FC | FC |
|---|---|---|
| name | (sAXL vs Epilife) | (DPFi CM vs Epilife) |
| EPHA4 | 14.921973 | 10.557523 |
| ATP6V0A2 | 11.761193 | 9.18081 |
| DIDO1 | 11.577912 | 11.624034 |
| FAM49B | 11.449174 | 10.309539 |
| SOS1 | 11.297932 | 12.510902 |
| MAP7D3 | 10.468552 | 13.538113 |
| GIT2 | 9.92446 | 10.028248 |
| SENP2 | 9.882794 | 10.513346 |
| DSCAM | 9.637356 | 8.835972 |
| NDUFAF4 | 9.1372 | 9.017144 |
| IL33 | −10.3360815 | −15.048633 |
| ICA1 | −10.473449 | −11.850388 |
| C7orf57 | −11.098472 | −8.344663 |
| LOC100129518///SOD2 | −11.761277 | −11.891947 |
| ADAM9 | −12.056555 | −10.755545 |
| OSMR | −12.578187 | −10.769784 |
| TOR1A | −14.24639 | −11.107677 |
| CCL20 | −15.549934 | −14.422149 |
| CDCP1 | 18.541739 | 22.558874 |
| IL13RA2 | −21.426365 | −22.007032 |

To determine if these genes would also be differentially regulated ex vivo, the inventors isolated RNA from the leading edge of the epidermis of the ex vivo punches treated with Epilife, sAXL or DPFi CM. Here, only the EphA4 results were able to be duplicated (FIG. 20), highlighting the Ephrin's pathway involvement in the wound healing process.

Conclusions

Not wishing to be bound to any particular hypothesis, in the context of a chronic wound where re-epithelisation is impaired, the inventors believe AXL will kick start the wound healing process, and thus promote closure where previously there was none. Chronic wounds have enhanced risk of wound site infection and therefore promoting re-epithelisation will also help to reduce infection. However, promoting re-epithelisation has advantages in other contexts, such as the reduction of scarring in normal wound closure and chronic wounds. Scarring is an inherent human property, which occurs due to impaired dermal re-modelling in the third phase of wound closure. However, chronic wounds with delayed re-epithelisation are characterised by extensive scarring, and there are clear links between scar formation and the time it takes for the wound to initially close. For example, re-epithelisation also occurs faster in oral wounds compared to skin wounds, and oral scars are few and far between. In vitro, oral keratinocytes migrate three times faster than skin keratinocytes in scratch wound assays. Thus, without wishing to be bound to any particular hypothesis, the inventors believe that targeting and accelerating the very first stage of wound healing, re-epithelisation, will have be useful both for the closure of chronic wounds, and in the reduction scar formation in the skin after injury.

In conclusion, and not wishing to be bound to any hypothesis, the inventors propose that the cleaved extracellular domain of AXL is a novel peptide which can be used to promote faster wound closure and reduce scarring of human skin by accelerating re-epithelisation.

### REFERENCES

1. R. R. Driskell, F. M. Watt, Understanding fibroblast heterogeneity in the skin. Trends Cell Biol 25, 92-99 (2015).

2. G. Sriram, P. L. Bigliardi, M. Bigliardi-Qi, Fibroblast heterogeneity and its implications for engineering organotypic skin models in vitro. Eur J Cell Biol 94, 483-512 (2015).

3. R. R. Driskell, C. A. B. Jahoda, C.-M. Chuong, F. M. Watt, V. Horsley, Defining dermal adipose tissue. Exp Dermatol 23, 629-631 (2014).

4. C. M. Alexander et al., Dermal white adipose tissue: a new component of the thermogenic response. Journal of lipid research 56, 2061-2069 (2015).

5. F. Gottrup, J. Apelqvist, P. Price, Outcomes in controlled and comparative studies on non-healing wounds: recommendations to improve the quality of evidence in wound management. J Wound Care 19, 237-268 (2010).

6. J. F. Guest et al., Health economic burden that wounds impose on the National Health Service in the UK. BMJ Open 5, e009283 (2015).

7. D. Upton, A. Andrews, Sleep disruption in patients with chronic leg ulcers. J Wound Care 22, 389-390, 392, 394 (2013).

8. D. Upton, P. Upton, R. Alexander, Well-being in wounds inventory (WOWI): development of a valid and reliable measure of well-being in patients with wounds. J Wound Care 25, 114, 116-120 (2016).

9. I. Pastar et al., Epithelialization in Wound Healing: A Comprehensive Review. Adv Wound Care (New Rochelle) 3, 445-464 (2014).

10. S. Werner, T. Krieg, H. Smola, Keratinocyte-fibroblast interactions in wound healing. J Invest Dermatol 127, 998-1008 (2007).

11. Y. Rinkevich et al., Identification and isolation of a dermal lineage with intrinsic fibrogenic potential. Science 348, (2015).

12. R. R. Driskell et al., Distinct fibroblast lineages determine dermal architecture in skin development and repair. Nature 504, 277-281 (2013).

13. N. Ojeh et al., The effects of caffeine on wound healing. Int Wound J 13, 605-613 (2016).

14. R. M. Gallucci, D. K. Sloan, J. M. Heck, A. R. Murray, S. J. O'Dell, Interleukin 6 indirectly induces keratinocyte migration. J Invest Dermatol 122, 764-772 (2004).

15. R. B. Mateo, J. S. Reichner, J. E. Albina, Interleukin-6 activity in wounds. Am J Physiol 266, R1840-1844 (1994).

16. J. P. O'Bryan et al., axl, a transforming gene isolated from primary human myeloid leukemia cells, encodes a novel receptor tyrosine kinase. Mol. Cell. Biol. 11, 5016-5031 (1991).

17. M. A. McDonnell, B. K. Law, R. Serra, H. L. Moses, Antagonistic effects of TGFbeta1 and BMP-6 on skin keratinocyte differentiation. Exp. Cell Res. 263, 265-273 (2001).

18. M. Rendl, L. Polak, E. Fuchs, BMP signaling in dermal papilla cells is required for their hair follicle-inductive properties. Genes Dev. 22, 543-557 (2008).

19. K. McGrory, C. M. Flaitz, J. R. Klein, Chemokine changes during oral wound healing. Biochem. Biophys. Res. Commun. 324, 317-320 (2004).

20. P. J. Finley, C. E. DeClue, S. A. Sell, J. M. DeBartolo, L. P. Shornick, Diabetic Wounds Exhibit Decreased Ymi and Arginase Expression with Increased Expression of IL-17 and IL-20. Adv Wound Care (New Rochelle) 5, 486-494 (2016).

21. G. Lemke, Biology of the TAM receptors. Cold Spring Harb. Perspect. Biol. 5, a009076 (2013).

22. P. Bellosta, M. Costa, D. A. Lin, C. Basilico, The receptor tyrosine kinase ARK mediates cell aggregation by homophilic binding. Mol. Cell. Biol. 15, 614-625 (1995).

23. V. A. Korshunov, Axl-dependent signalling: a clinical update. Clin. Sci. (Lond.) 122, 361-368 (2012).

24. J. Mendoza-Garcia, A. Sebastian, T. Alonso-Rasgado, A. Bayat, Optimization of an ex vivo wound healing model in the adult human skin: Functional evaluation using photodynamic therapy. Wound Repair Regen 23, 685-702 (2015).

25. Borowicz, S., M. Van Scoyk, S. Avasarala, M. K. Karuppusamy Rathinam, J. Tauler, R. K. Bikkavilli and R. A. Winn (2014). “The soft agar colony formation assay.” J Vis Exp (92): e51998.

26. Meilang Xue, Christopher J. Jackson. Extracellular Matrix Reorganization During Wound Healing and Its Impact on Abnormal Scarring. Advances in Wound Care 4, 119-136 Mary Ann Liebert Inc, 2015.

27. Rong Yan-hua Wang Cheng, Zhang Guo-an. The content and ratio of type I and III collagen in skin differ with age and injury |Cheng| African Journal of Biotechnology. African Journal of Biotechnology 10 (2011).

1. A method of treating a wound, or of preventing, reducing or inhibiting scar formation, the method comprising administering, to a subject in need thereof, a therapeutic amount of a polypeptide selected from a group consisting of AXL, CCL19 and BMP-6, or a biologically active variant or fragment thereof.

2. The method to claim 1, wherein the polypeptide is AXL or a biologically active variant or fragment thereof comprising the active domain of AXL, and optionally the polypeptide is soluble AXL, and optionally further comprising administration of CCL19, or a biologically active variant or fragment thereof and/or BMP-6, or a biologically active variant or fragment thereof.

3. (canceled)

4. (canceled)

5. The method to claim 1, wherein the polypeptide is CCL19, or a biologically active variant or fragment thereof.

6. The method to claim 1, wherein the polypeptide is BMP-6, or a biologically active variant or fragment thereof.

7. The method to claim 1, wherein the polypeptide is substantially as set out in SEQ ID NO: 1 or a variant or fragment thereof, and optionally as set out in SEQ ID NO: 3 or a variant or fragment thereof.

8. The method to claim 1, wherein the polypeptide is encoded by the nucleotide sequence substantially as set out in SEQ ID NO: 2 or a variant or fragment thereof, and optionally substantially as set out in SEQ ID NO: 4 or a variant or fragment thereof.

9. The method to claim 1, wherein the polypeptide is substantially as set out in SEQ ID NO: 10 or a variant or fragment thereof.

10. The method to claim 1, wherein the polypeptide is encoded by the nucleotide sequence substantially as set out in SEQ ID NO: 11 or a variant or fragment thereof.

11. The method to claim 1, wherein the polypeptide is substantially as set out in SEQ ID NO: 12 or a variant or fragment thereof.

12. The method to claim 1, wherein the polypeptide is encoded by the nucleotide sequence as set out in SEQ ID NO: 13 or a variant or fragment thereof.

13. The method to claim 1, wherein the treatment comprises re-epithelisation of epithelial tissue, and optionally wherein the rate of wound healing is increased and/or scar formation is prevented, reduced or inhibited.

14. (canceled)

15. The method to claim 1, wherein the wound is present on the skin.

16. (canceled)

17. A method of treating a wound, the method comprising administering, to a subject in need thereof, a therapeutic amount of a vector comprising a nucleic acid encoding a polypeptide selected from a group consisting of AXL, CCL19 and BMP-6, or a biologically active variant or fragment thereof.

18. (canceled)

19. A method according to claim 1, wherein the polypeptide is in a pharmaceutically acceptable composition, and the composition further comprises a pharmaceutically acceptable vehicle.

20. (canceled)

21. (canceled)

22. A device comprising a polypeptide selected from the group consisting of AXL, CCL19 and BMP-6, or a biologically active variant or fragment thereof, wherein the device is configured for the controlled spatio-temporal delivery of the polypeptide.

23. The device according to claim 22, wherein the device is a bandage comprising at least two layers comprising a polypeptide selected from the group consisting of AXL, CCL19 and BMP-6, or a biologically active variant or fragment thereof, wherein each layer comprises the same or different polypeptide, wherein the polypeptide comprised in a different layer is delivered to the wound site at a different time point, and optionally wherein the polypeptide is CCL19 in one layer and AXL in another layer, and CCL19 is delivered first, optionally for up to 2 days, and AXL is delivered after CCL19, optionally for the remainder of wound closure.

24. (canceled)

25. (canceled)

26. (canceled)

27. (canceled)

28. (canceled)

29. (canceled)

30. (canceled)


SEQUENCE LISTING (14 sequences)

SEQ ID NO: 1 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 2 - DNA, 2685 bp, Homo sapiens
atggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctgagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagggcaagcacagccagtccaccagctggtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcctgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcggggaccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccagggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctga

SEQ ID NO: 3 - protein, 417 aa, Homo sapiens
EESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWP

SEQ ID NO: 4 - DNA, 1247 bp, Homo sapiens
aaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctgagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagggcaagcacagccagtccaccagctggtgaaggaaccttcaactcctgccttctcgtggccc

SEQ ID NO: 5 - protein, 885 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 6 - protein, 88 aa, Homo sapiens
FVGNPGNITGARGLTGTLRCQLQVQGEPPEVHWLRDGQILELADSTQTQVPLGEDEQDDWIVVSQLRITSLQLSDTGQYQCLVFLGHQ

SEQ ID NO: 7 - protein, 72 aa, Homo sapiens
FLEEPEDRTVAANTPFNLSCQAQGPPEPVDLLWLQDAVPLATAPGHGPQRSLHVPGLNKTSSFSCEAHNAKG

SEQ ID NO: 8 - protein, 99 aa, Homo sapiens
LPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTH

SEQ ID NO: 9 - protein, 104 aa, Homo sapiens
PVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRP

SEQ ID NO: 10 - protein, 104 aa, Homo sapiens
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKASLALPGPVSSL

SEQ ID NO: 11 - DNA, 315 bp, Homo sapiens
atggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaaggcaagcctggccctccctggccctgtctcctccctctga

SEQ ID NO: 12 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 13 - DNA, 1542 bp, Homo sapiens
atgccggggctggggcggagggcgcagtggctgtgctggtggtgggggctgctgtgcagctgctgcgggcccccgccgctgcggccgcccttgcccgctgccgcggccgccgccgccggggggcagctgctgggggacggcgggagccccggccgcacggagcagccgccgccgtcgccgcagtcctcctcgggcttcctgtaccggcggctcaagacgcaggagaagcgggagatgcagaaggagatcttgtcggtgctggggctcccgcaccggccccggcccctgcacggcctccaacagccgcagcccccggcgctccggcagcaggaggagcagcagcagcagcagcagctgcctcgcggagagccccctcccgggcgactgaagtccgcgcccctcttcatgctggatctgtacaacgccctgtccgccgacaacgacgaggacggggcgtcggagggggagaggcagcagtcctggccccacgaagcagccagctcgtcccagcgtcggcagccgcccccgggcgccgcgcacccgctcaaccgcaagagccttctggcccccggatctggcagcggcggcgcgtccccactgaccagcgcgcaggacagcgccttcctcaacgacgcggacatggtcatgagctttgtgaacctggtggagtacgacaaggagttctcccctcgtcagcgacaccacaaagagttcaagttcaacttatcccagattcctgagggtgaggtggtgacggctgcagaattccgcatctacaaggactgtgttatggggagttttaaaaaccaaacttttcttatcagcatttatcaagtcttacaggagcatcagcacagagactctgacctgtttttgttggacacccgtgtagtatgggcctcagaagaaggctggctggaatttgacatcacggccactagcaatctgtgggttgtgactccacagcataacatggggcttcagctgagcgtggtgacaagggatggagtccacgtccacccccgagccgcaggcctggtgggcagagacggcccttacgacaagcagcccttcatggtggctttcttcaaagtgagtgaggtgcacgtgcgcaccaccaggtcagcctccagccggcgccgacaacagagtcgtaatcgctctacccagtcccaggacgtggcgcgggtctccagtgcttcagattacaacagcagtgaattgaaaacagcctgcaggaagcatgagctgtatgtgagtttccaagacctgggatggcaggactggatcattgcacccaagggctatgctgccaattactgtgatggagaatgctccttcccactcaacgcacacatgaatgcaaccaaccacgcgattgtgcagaccttggttcaccttatgaaccccgagtatgtccccaaaccgtgctgtgcgccaactaagctaaatgccatctcggttctttactttgatgacaactccaatgtcattctgaaaaaatacaggaatatggttgtaagagcttgtggatgccactaa

SEQ ID NO: 14 - protein, 9 aa, Homo sapiens
GQAQPVHQL